(19) United States
(12) Patent Application Publication
(10) Pub. No.: US 2024/0180855 A1
(43) Pub. Date: Jun. 6, 2024

(54) BIOACTIVE PRODUCTS

(71) Applicant: Victor TETS, New York, NY (US)

(72) Inventors: Victor TETS, New York, NY (US); Georgy TETS, New York, NY (US)

(21) Appl. No.: 18/285,641

(22) PCT Filed: Apr. 5, 2022
(86) PCT No.: PCT/IB2022/053167

§ 371 Date: Oct. 4, 2023

Related U.S. Application Data
(60) Provisional application No. 63/170,822, filed on Apr. 5, 2021.

(51) Int. Cl. A61K 31/155; A01N 43/54; A01N 43/82; A01N 47/28; A01N 47/44; A01N 59/16; A01N 63/50; A01P 3/00; A01P 21/00; A61K 33/30; A61K 47/02; A61K 47/10; A61K 47/18; A61K 47/26; A61K 47/36; A61P 1/02; A61P 11/02; A61P 13/10; A61P 17/00; A61P 17/02; A61P 17/06; A61P 17/08; A61P 27/02; A61P 31/04; A61P 31/10; A61P 31/22; C02F 1/50; C02F 103/20

(52) U.S. Cl. CPC A61K 31/155; A01N 43/54; A01N 43/82; A01N 47/28; A01N 47/44; A01N 59/16; A01N 63/50; A01P 3/00; A01P 21/00; A61K 33/30; A61K 47/02; A61K 47/10; A61K 47/183; A61K 47/26; A61K 47/36; A61P 1/02; A61P 11/02; A61P 13/10; A61P 17/00; A61P 17/02; A61P 17/06; A61P 17/08; A61P 27/02; A61P 31/04; A61P 31/10; A61P 31/22; C02F 1/50; C02F 2103/20

(57) ABSTRACT

The present invention relates to the compounds and methods for controlling living organisms for their bioactivity, improving their habitat in order to increase the biological activity of living beings in various conditions, as well as the prevention and treatment of diseases of plants, animals and humans.

### FIELD OF THE INVENTION

The present invention relates to the compounds and methods for controlling living organisms for their bioactivity, adaptation to various conditions, prevention and treatment of diseases, modulation of synthetic activity and product yield in animals, insects, plants.

### BACKGROUND OF THE INVENTION

Known protein receptors, action on which can influence the course of cancer or work of the immune system. However, the available products are limited by the specificity of protein receptors and have a limited and narrowly targeted effect. The previously unknown Tetz receptor system, discovered by us, is universal for cells and communities of prokaryotes, as well as cells, tissues and organs of eukaryotes and is formed by special molecules of nucleic acids. The Tetz system controls the interaction of living beings with any chemical, physical and biological factors of the environment.

The impact on the components of the Tetz system makes it relevant to achieve unexpected possibilities with the help of various molecules to change the behavior of living organisms. Such products will make it possible to achieve previously impossible results, which are of great practical importance in crop production, animal husbandry, fish farming to increase productivity, as well as the prevention and treatment of diseases of plants, animals and humans.

Among the various chemical compounds with biological activity, the product M4 is known, which has an antimicrobial effect, inhibitors of viral integrases and reverse transcriptases, viral proteases, DNases and RNases, but their direct bioactive effects.

### SUMMARY OF THE INVENTION

Various non-limiting aspects and embodiments of the invention are described below.

In some embodiments invention, the product is a complex of DNase (from 0.1 μg/ml to 500.0 μg/ml), and/or RNAse (from 0.1 μg/ml to 500.0 μg/ml), and/or DNase+RNAse (from 0.1 μg/ml up to 500.0 μg/ml) and/or reverse transcription inhibitors (from 0.1 μg/ml to 5000.0 μg/ml), and/or integration/recombination (from 0.1 μg/ml to 5000.0 μg/ml), and/or proteases (from 0.1 μg/ml to 5000.0 μg/ml), as well as their forms, which are gels and/or emulsions and/or ointments and/or solutions, intended for the prevention and treatment of animals and humans.

In some embodiments invention, the products are gels and/or emulsions and/or ointments, additionally including hydrophilic ointment bases, including lightly crosslinked acrylic polymers, and/or lipophilic hydrocarbon, fatty, silicone and other components

In some embodiments invention, the product is M4 in an amount from 0.001 μg/ml to 10e5 μg/ml or compositions of M4 and zinc salts (from 0.1 μg/ml to 5000.0 μg/ml) of glycerol (from 0.1 μg/ml to 5000.0 μg/ml) sorbitol (from 0.1 μg/ml to 5000.0 μg/ml), boron salts (from 0.1 μg/ml to 5000.0 μg/ml), glutamate salts (from 0.1 μg/ml to 5000.0 μg/ml), mannitol (from 0.1 μg/ml to 5000.0 μg/ml), salts of hydrogen phosphates (from 0.1 μg/ml to 5000.0 μg/ml), salts of dihydrophosphates (from 0.1 μg/ml to 5000.0 μg/ml) salts of manganese (from 0.1 μg/ml to 5000.0 μg/ml), glutamic acid salts (from 0.1 μg/ml to 5000.0 μg/ml), or reverse transcription inhibitors (from 0.1 μg/ml to 5000.0 μg/ml), integration/recombination (from 0.1 μg/ml to 5000.0 μg/ml), proteases (from 0.1 μg/ml to 5000.0 μg/ml), DNase (from 0.1 μg/ml to 500.0 μg/ml), RNAse (from 0.1 μg/ml to 500.0 μg/ml), DNase+RNAse (from 0.1 μg/ml to 500.0 μg/ml) and complexes of the listed products, which are used to increase agricultural productivity natural and/or ornamental and/or forest and/or domestic plants and/or aquaculture.

In some embodiments invention, when the product is a M4 complex in an amount from 0.001 μg/ml to 10e5 μg/ml and zinc salt (from 0.1 μg/ml to 5000.0 μg/ml), and/or glycerol (from 0.1 μg/ml to 5000.0 μg/ml) sorbitol (from 0.1 μg/ml to 5000.0 μg/ml), boron salts (from 0.1 μg/ml to 5000.0 μg/ml), glutamate salts and/or (from 0.1 μg/ml to 5000.0 μg/ml), and/or mannitol (from 0.1 μg/ml to 5000.0 μg/ml), and/or salts of hydrogen phosphates (from 0.1 μg/ml to 5000.0 μg/ml), and/or salts of dihydrogen phosphates (from 0.1 μg/ml to 5000.0 μg/ml) and/or manganese salts (from 0.1 μg/ml to 5000.0 μg/ml), and/or glucuronic acid soda (from 0.1 μg/ml to 5000.0 μg/ml) which is used to increase the productivity of agricultural and/or decorative and/or forest and/or domestic plants and/or aquaculture by seed dressing and/or treatment of roots and/or vegetative parts of the plant.

In some embodiments invention, the product is a complex of M4 in an amount from 0.001 μg/ml to 10e5 μg/ml, zinc salts (from 0.1 μg/ml to 5000.0 μg/ml) and DNAse (from 0.1 μg/ml to 500.0 μg/ml), which is used for the purpose of increasing the productivity of agricultural and/or ornamental and/or forest and/or domestic plants and/or aquaculture by treating seeds and/or treating the roots and/or vegetative parts of the plant.

In some embodiments invention, the product is a complex of M4 in an amount from 0.001 μg/ml to 10e5 μg/ml, zinc salts (from 0.1 μg/ml to 5000.0 μg/ml) and RNAse (from 0.1 μg/ml to 500.0 μg/ml), which is used to increase the productivity of agricultural and/or ornamental and/or forest and/or domestic plants and/or aquaculture due to seed dressing and/or treatment of roots and/or vegetative parts of the plant.

In some embodiments invention, the product is a complex of M4 in an amount from 0.001 μg/ml to 10e5 μg/ml, zinc salts (from 0.1 μg/ml to 5000.0 μg/ml), DNAse (from 0.1 μg/ml to 500.0 μg/ml) and RNAse (from 0.1 μg/ml to 500.0 μg/ml), which is used to increase the productivity of agricultural and/or ornamental and/or forest and/or domestic plants and/or aquaculture.

In some embodiments invention, the product is a complex of M4 in an amount from 0.001 μg/ml to 10e5 μg/ml, zinc salts (from 0.1 μg/ml to 5000.0 μg/ml) and DNAse (from 0.1 μg/ml to 500.0 μg/ml) and Raltegravir (from 0.1 μg/ml to 5000.0 μg/ml), which is used to increase the productivity of agricultural and/or ornamental and/or forest and/or domestic plants and/or aquaculture by seed treatment and/or root treatment and/or vegetative parts of the plant.

In the In some embodiments invention, the product is a complex of DNAse (from 0.1 μg/ml to 500.0 μg/ml) or RNAse (from 0.1 μg/ml to 500.0 μg/ml) or DNAse mRNAse (from 0.1 μg/ml to 500.0 μg/ml) and Raltegravir (from 0.1 μg/ml to 5000.0 μg/ml), which is used to increase the productivity of agricultural and/or ornamental and/or forest and/or domestic plants and/or aquaculture by seed treatment and/or root treatment and/or vegetative parts of the plant.

In some embodiments invention, the product is M4 in an amount from 0.001 μg/ml to 10e5 μg/ml or compositions of M4 and zinc salts (from 0.1 μg/ml to 5000.0 μg/ml) of glycerol (from 0.1 μg/ml to 5000.0 μg/ml) sorbitol (from 0.1 μg/ml to 5000.0 μg/ml), boron salts (from 0.1 μg/ml to 5000.0 μg/ml), glutamate salts (from 0.1 μg/ml to 5000.0 μg/ml), mannitol (from 0.1 μg/ml to 5000.0 μg/ml), salts of hydrogen phosphates (from 0.1 μg/ml to 5000.0 μg/ml), salts of dihydrophosphates (from 0.1 μg/ml to 5000.0 μg/ml) salts of manganese (from 0.1 μg/ml to 5000.0 μg/ml), glutamic acid salts (from 0.1 μg/ml to 5000.0 μg/ml), or reverse transcription inhibitors (from 0.1 μg/ml to 5000.0 μg/ml), integration/recombination (from 0.1 μg/ml to 5000.0 μg/ml), proteases (from 0.1 μg/ml to 5000.0 μg/ml), DNAse (from 0.1 μg/ml to 500.0 μg/ml), RNAse (from 0.1 μg/ml to 500.0 μg/ml), DNAse+RNAse (from 0.1 μg/ml to 500.0 μg/ml) and complexes of the listed products that are used to process fish eggs in order to increase the effective fertilization, sex management and infection control.

In some embodiments invention, the product is M4 in an amount from 0.001 μg/ml to 10e5 μg/ml or compositions of M4 and zinc salts (from 0.1 μg/ml to 5000.0 μg/ml), glycerol (from 0.1 μg/ml to 5000.0 μg/ml), sorbitol (from 0.1 μg/ml to 5000.0 μg/ml), boron salts (from 0.1 μg/ml to 5000.0 μg/ml), glutamate salts (from 0.1 μg/ml to 5000.0 μg/ml), mannitol (from 0.1 μg/ml to 5000.0 μg/ml), salts of hydrogen phosphates (from 0.1 μg/ml to 5000.0 μg/ml), salts of dihydrophosphates (from 0.1 μg/ml to 5000.0 μg/ml) salts of manganese (from 0.1 μg/ml to 5000.0 μg/ml), salts of glutamic acid (from 0.1 μg/ml to 5000.0 μg/ml), and complexes of the listed products that are used to increase the growth rate of weight gain, and other vital and commercially important characteristics of animals and aquaplankton through feed processing.

In some embodiments invention, the product is M4 in an amount from 0.001 μg/ml to 10e5 μg/ml or compositions of M4 and zinc salts (from 0.1 μg/ml to 5000.0 μg/ml), glycerol (from 0.1 μg/ml to 5000.0 μg/ml), sorbitol (from 0.1 μg/ml to 5000.0 μg/ml), boron salts (from 0.1 μg/ml to 5000.0 μg/ml), glutamate salts (from 0.1 μg/ml to 5000.0 μg/ml), mannitol (from 0.1 μg/ml to 5000.0 μg/ml), salts of hydrogen phosphates (from 0.1 μg/ml to 5000.0 μg/ml), salts of dihydrogen phosphates (from 0.1 μg/ml to 5000.0 μg/ml) of manganese salts (from 0.1 μg/ml to 5000.0 μg/ml), glutamic acid salts (from 0.1 μg/ml to 5000.0 μg/ml), or reverse transcription inhibitors (from 0.1 μg/ml to 5000.0 μg/ml), integration/recombination (from 0.1 μg/ml to 5000.0 μg/ml), proteases (from 0.1 μg/ml to 5000.0 μg/ml), DNAse (from 0.1 μg/ml to 500.0 μg/ml), RNAse (from 0.1 μg/ml to 500.0 μg/ml), DNAse+RNAse (from 0.1 μg/ml to 500.0 μg/ml) of the complexes of the listed products, as well as their forms, which are gels and/or emulsions and/or ointments and/slugs and solutions that are used to treat eye diseases with conjunctivitis and dacryocystitis.

In some embodiments invention, the product is a complex of DNAse (from 0.1 μg/ml to 500.0 μg/ml), RNAse (from 0.1 μg/ml to 500.0 μg/ml), DNAse+RNAse (from 0.1 μg/ml to 500.0 μg/ml) or inhibitors of reverse transcription (from 0.1 μg/ml to 5000.0 μg/ml), integration/recombination (from 0.1 μg/ml to 5000.0 μg/ml), proteases (from 0.1 μg/ml to 5000.0 μg/ml), as well as their forms, which are gels and/or emulsions and/or ointments and/or solutions that are used to treat eye diseases with conjunctivitis and dacryocystitis.

In some embodiments invention, the product is M4 in an amount from 0.001 μg/ml to 10e5 μg/ml or compositions of M4 and zinc salts (from 0.1 μg/ml to 5000.0 μg/ml) of glycerol (from 0.1 μg/ml to 5000.0 μg/ml) sorbitol (from 0.1 μg/ml to 5000.0 μg/ml), boron salts (from 0.1 μg/ml to 5000.0 μg/ml), glutamate salts (from 0.1 μg/ml to 5000.0 μg/ml), mannitol (from 0.1 μg/ml to 5000.0 μg/ml), salts of hydrogen phosphates (from 0.1 μg/ml to 5000.0 μg/ml), salts of dihydrophosphates (from 0.1 μg/ml to 5000.0 μg/ml) salts of manganese (from 0.1 μg/ml to 5000.0 μg/ml), glutamic acid salts (from 0.1 μg/ml to 5000.0 μg/ml), or reverse transcription inhibitors (from 0.1 μg/ml to 5000.0 μg/ml), integration/recombination (from 0.1 μg/ml to 5000.0 μg/ml), proteases (from 0.1 μg/ml to 5000.0 g/ml), DNAse (from 0.1 μg/ml to 500.0 μg/ml), RNAse (from 0.1 μg/ml to 500.0 g/ml). DNAse+RNAse (from 0.1 μg/ml to 500.0 μg/ml) and complexes of the listed drugs, which are gels and/or emulsions and/or ointments and/or solutions of co which are used to correct the condition of the oral cavity, including periodontal and endodontic mucosa, as well as cysts and granulomas.

In some embodiments invention, the product is M4 in an amount from 0.001 μg/ml to 10e5 μg/ml or compositions of M4 and zinc salts (from 0.1 μg/ml to 5000.0 μg/ml) of glycerol (from 0.1 μg/ml to 5000.0 μg/ml) sorbitol (from 0.1 μg/ml to 5000.0 μg/ml), boron salts (from 0.1 μg/ml to 5000.0 μg/ml), glutamate salts (from 0.1 μg/ml to 5000.0 μg/ml), mannitol (from 0.1 μg/ml to 5000.0 μg/ml), salts of hydrogen phosphates (from 0.1 μg/ml to 5000.0 μg/ml), salts of dihydrophosphates (from 0.1 μg/ml to 5000.0 μg/ml) salts of manganese (from 0.1 μg/ml to 5000.0 μg/ml), glutamic acid salts (from 0.1 μg/ml to 5000.0 μg/ml), or reverse transcription inhibitors (from 0.1 μg/ml to 5000.0 μg/ml), integration/recombination (from 0.1 μg/ml to 5000.0 μg/ml), proteases (from 0.1 μg/ml to 5000.0 μg/ml), DNAse (from 0.1 μg/ml to 500.0 μg/ml), RNAse (from 0.1 μg/ml to 500.0 μg/ml), DNAse+RNAse (from 0.1 μg/ml to 500.0 μg/ml) and complexes of the listed drugs that are used to correct the condition of the skin, subcutaneous cells atki and/or eyes and/or mucous membranes of animals and humans with various diseases, including seborrheic dermatitis, neuroderma Psoriasis, Herpes, papillomatosis, mycoses, erysipelas, trophic and diabetic ulcers, trauma, acne bedsores, folliculitis, furunculosis, angular cheilitis (angulitis), alopecia.

In the In some embodiments invention, the product is M4 in an amount from 0.001 μg/ml to 10e5 μg/ml or compositions of M4 and zinc salts (from 0.1 μg/ml to 5000.0 μg/ml) of glycerol (from 0.1 μg/ml to 5000.0 μg/ml) sorbitol (from 0.1 μg/ml to 5000.0 μg/ml), boron salts (from 0.1 μg/ml to 5000.0 μg/ml), glutamate salts (from 0.1 μg/ml to 5000.0 μg/ml), mannitol (from 0.1 μg/ml to 5000.0 μg/ml), salts of hydrogen phosphates (from 0.1 μg/ml to 5000.0 μg/ml), salts of dihydrophosphates (from 0.1 μg/ml to 5000.0 μg/ml) salts of manganese (from 0.1 μg/ml to 5000.0 μg/ml), glutamic acid salts (from 0.1 μg/ml to 5000.0 μg/ml), or reverse transcription inhibitors (from 0.1 μg/ml to 5000.0 μg/ml), integration/recombination (from 0.1 μg/ml to 5000.0 μg/ml), proteases (from 0.1 μg/ml to 5000.0 μg/ml), DNAse (from 0.1 μg/ml to 500.0 μg/ml), RNAse (from 0.1 μg/ml to 500.0 μg/ml), DNAse+RNAse (from 0.1 μg/ml to 500.0 μg/ml) and complexes of the listed drugs that are used to correct the condition of the sinuses and/or mucous membranes Bladder

In the In some embodiments invention, the product is M4 in an amount from 0.001 μg/ml to 10e5 μg/ml or compositions of M4 and zinc salts (from 0.1 μg/ml to 5000.0 μg/ml), glycerol (from 0.1 μg/ml to 5000.0 μg/ml), sorbitol (from 0.1 μg/ml to 5000.0 μg/ml), boron salts (from 0.1 μg/ml to 5000.0 μg/ml), glutamate salts (from 0.1 μg/ml to 5000.0 μg/ml), mannitol (from 0.1 μg/ml to 5000.0 μg/ml), salts of hydrophosphates (from 0.1 μg/ml to 5000.0 μg/ml), salts of dihydrophosphates (from 0.1 μg/ml to 5000.0 μg/ml) salts of manganese (from 0.1 μg/ml to 5000.0 μg/ml), glutamic acid salts (from 0.1 μg/ml to 5000.0 μg/ml), or reverse transcription inhibitors (from 0.1 μg/ml to 5000.0 g/ml), integration/recombination (from 0.1 μg/ml to 5000.0 μg/ml), proteases (from 0.1 μg/ml to 5000.0 μg/ml), DNAse (from 0.1 μg/ml to 500.0 μg/ml), RNAse (from 0.1 μg/ml to 500.0 μg/ml), DNAse+RNAse (from 0.1 μg/ml to 500.0 μg/ml) of the complexes of the listed drugs, as well as their forms, which are gels and/or emulsions and/or ointments and/and whether solutions that are used to prevent and treat hoof rot and/or animal skin diseases

In some embodiments invention, a method is provided for increasing germination, intensity and growth rate, chlorophyll formation and productivity of plants, increasing the productivity of aquaculture, fertilizing fish, processing soil, water, breeding aquatic animals, and aquariums, increasing the safety of feed for farm animals and aquaculture, prevention and treatment diseases and condition management (or condition correction) of plants, animals and people.

In some embodiments invention provides a method in which soil and/or water bodies are treated with M4 product in an amount from 0.001 μg/ml to 10e5 μg/ml in order to increase the productivity of agricultural and/or ornamental and/or forest and/or domestic plants and/or aquaculture or compositions M4 and zinc salts (from 0.1 μg/ml to 5000.0 μg/ml) glycerol (from 0.1 μg/ml to 5000.0 μg/ml) sorbitol (from 0.1 μg/ml to 5000.0 μg/ml), boron salts (from 0.1 μg/ml to 5000.0 μg/ml), glutamate salts (from 0.1 μg/ml to 5000.0 μg/ml), mannitol (from 0.1 μg/ml to 5000.0 μg/ml), hydrogen phosphate salts (from 0.1 μg/ml to 5000.0 μg/ml), salts of dihydrophosphates (from 0.1 μg/ml to 5000.0 μg/ml) of manganese salts (from 0.1 μg/ml to 5000.0 μg/ml), the exposure time is from 10 seconds to 24 hours and after exposure, the drug is inactivated by adding carboxymethyl cellulose and/or sodium alginate in a ratio with the drug 0.5-1.0 to 100.0-1.0

In some embodiments invention provides a method in which soil and/or water bodies are treated with M4 product in an amount from 0.001 μg/ml to 10e5 μg/ml in order to increase the productivity of agricultural and/or ornamental and/or forest and/or domestic plants and/or aquaculture or compositions M4 and zinc salts (from 0.1 μg/ml to 5000.0 μg/ml) glycerol (from 0.1 μg/ml to 5000.0 μg/ml) sorbitol (from 0.1 μg/ml to 5000.0 μg/ml), boron salts (from 0.1 μg/ml to 5000.0 μg/ml), glutamate salts (from 0.1 μg/ml to 5000.0 μg/ml), mannitol (from 0.1 μg/ml to 5000.0 μg/ml), hydrogen phosphate salts (from 0.1 μg/ml to 5000.0 μg/ml), salts of dihydrophosphates (from 0.1 μg/ml to 5000.0 μg/ml) salts of manganese (from 0.1 μg/ml to 5000.0 μg/ml), the exposure time is from 10 seconds to 24 hours, the drug remains without inactivation for an unlimited time

In some embodiments invention provides a method in which reservoirs with running water are treated with drugs and the addition of the drug ensures that the required final concentration of the drug M4 is maintained in an amount from 0.001 μg/ml to 10e5 μg/ml or compositions M4 and zinc salts (from 0.1 μg/ml up to 5000.0 μg/ml) glycerol (from 0.1 μg/ml to 5000.0 μg/ml) sorbitol (from 0.1 μg/ml to 5000.0 μg/ml), boron salts (from 0.1 μg/ml to 5000.0 μg/ml), glutamate salts (from 0.1 μg/ml to 5000.0 μg/ml), mannitol (from 0.1 μg/ml to 5000.0 μg/ml), salts of hydrogen phosphates (from 0.1 μg/ml to 5000.0 μg/ml), salts of dihydrogen phosphates (from 0.1 μg/ml up to 5000.0 μg/ml) manganese salts (from 0.1 μg/ml to 5000.0 μg/ml),

In some embodiments invention provides a method for influencing plants by root and/or non-root method and/or by spraying for applying to the surface of vegetative shoots—leaves and stems and/or hydroponics and/or fertigation, using the product M4 in an amount of 0.001 μg/ml up to 10e5 μg/ml or compositions M4 and zinc salts (from 0.1 μg/ml to 5000.0 μg/ml) glycerol (from 0.1 μg/ml to 5000.0 μg/ml) sorbitol (from 0.1 μg/ml to 5000.0 μg/ml), boron salts (from 0.1 μg/ml to 5000.0 μg/ml), glutamate salts (from 0.1 μg/ml to 5000.0 μg/ml), mannitol (from 0.1 μg/ml to 5000.0 μg/ml), hydrogen phosphate salts (from 0.1 μg/ml to 5000.0 μg/ml), dihydrophosphate salts (from 0.1 μg/ml to 5000.0 μg/ml) manganese salts (from 0.1 μg/ml to 5000.0 μg/ml),

In some embodiments invention provides a method for influencing plants by root and/or non-root method and/or by spraying for applying to the surface of vegetative shoots—leaves and stems and/or hydroponics and/or fertigation, using the product M4 in an amount of 0.001 μg/ml up to 10e5 μg/ml or compositions M4 and zinc salts (from 0.1 μg/ml to 5000.0 μg/ml) glycerol (from 0.1 μg/ml to 5000.0 μg/ml) sorbitol (from 0.1 μg/ml to 5000.0 μg/ml), boron salts (from 0.1 μg/ml to 5000.0 μg/ml), glutamate salts (from 0.1 μg/ml to 5000.0 μg/ml), mannitol (from 0.1 μg/ml to 5000.0 μg/ml), hydrogen phosphate salts (from 0.1 μg/ml to 5000.0 μg/ml), dihydrophosphate salts (from 0.1 μg/ml to 5000.0 μg/ml) manganese salts (from 0.1 μg/ml to 5000.0 μg/ml), followed by inactivation with carboxymethyl cellulose, sodium alginate in relation to the drug 0.5-1.0 up to 100.0-1.0

In some embodiments invention, a method is provided for influencing eggs, to increase the efficiency of fertilization, and sex control, M4 product is used in an amount from 0.001 μg/ml to 10e5 μg/ml or compositions M4 and zinc salts (from 0.1 μg/ml to 5000.0 μg/ml) glycerin (from 0.1 μg/ml to 5000.0 μg/ml) sorbitol (from 0.1 μg/ml to 5000.0 μg/ml), boron salts (from 0.1 μg/ml to 5000.0 μg/ml), glutamate salts (from 0.1 μg/ml to 5000.0 μg/ml), mannitol (from 0.1 μg/ml to 5000.0 μg/ml), salts of hydrogen phosphates (from 0.1 μg/ml to 5000.0 μg/ml), salts of dihydrogen phosphates (from 0.1 μg/ml to 5000.0 μg/ml) manganese salts (from 0.1 μg/ml to 5000.0 μg/ml), or reverse transcription inhibitors (from 0.1 μg/ml to 5000.0 μg/ml), integration/recombination (from 0.1 μg/ml to 5000.0 μg/ml), proteases (from 0.1 μg/ml to 5000.0 μg/ml), DNAse (from 0.1 μg/ml to 500.0 μg/ml), RNAse (from 0.1 μg/ml to 500.0 μg/ml), DNAse+RNAse (from 0.1 μg/ml to 500.0 μg/ml) and complexes of the listed drugs, and the treatment continues from 3 seconds to 1 hour

In some embodiments invention provides a method for influencing fish and/or crustaceans and/or molluscs to increase productivity, using M4 product in an amount from 0.001 μg/ml to 10e5 μg/ml or compositions M4 and zinc salts (from 0.1 μg/ml to 5000.0 μg/ml) glycerol (from 0.1 μg/ml to 5000.0 μg/ml) sorbitol (from 0.1 μg/ml to 5000.0 μg/ml), boron salts (from 0.1 μg/ml to 5000.0 μg/ml), glutamate salts (from 0.1 μg/ml to 5000.0 μg/ml), mannitol (from 0.1 μg/ml to 5000.0 μg/ml), salts of hydrogen phosphates (from 0.1 μg/ml to 5000.0 μg/ml), salts of dihydrogen phosphates (from 0.1 μg/ml to 5000.0 μg/ml) of manganese salts (from 0.1 μg/ml to 5000.0 μg/ml), and the treatment lasts from 3 seconds to 24 hours

In some embodiments invention provides a method for influencing fish and/or crustaceans and/or molluscs to increase productivity, using M4 product in an amount from 0.001 μg/ml to 10e5 μg/ml or compositions M4 and zinc salts (from 0.1 μg/ml to 5000.0 g/ml) glycerol (from 0.1 μg/ml to 5000.0 μg/ml) sorbitol (from 0.1 μg/ml to 5000.0 μg/ml), boron salts (from 0.1 μg/ml to 5000.0 μg/ml), glutamate salts (from 0.1 μg/ml to 5000.0 μg/ml), mannitol (from 0.1 μg/ml to 5000.0 μg/ml), salts of hydrogen phosphates (from 0.1 μg/ml to 5000.0 μg/ml), salts of dihydrogen phosphates (from 0.1 μg/ml to 5000.0 μg/ml) of manganese salts (from 0.1 μg/ml to 5000.0 μg/ml), is introduced into the water where the fish are located and remains there indefinitely and/or is inactivated by carboxymethyl cellulose, sodium alginate in a ratio with the drug 0.5-1, 0 to 100.0-1.0

In a In some embodiments invention, a method is provided for influencing fish and/or crustaceans and/or molluscs to increase productivity, which includes treating them with a product and releasing them into water, also pretreated with the product.

In a In some embodiments invention, a method is provided in which to increase the growth rate, body weight gain, and other vital and commercially important characteristics of animals and aquaplankton, the feed is treated before feeding using the product M4 in an amount from 0.001 μg/ml to 10e5 μg/ml or compositions M4 and zinc salts (from 0.1 μg/ml to 5000.0 μg/ml) glycerin (from 0.1 μg/ml to 5000.0 μg/ml) sorbitol (from 0.1 μg/ml to 5000.0 μg/ml), boron salts (from 0.1 μg/ml to 5000.0 μg/ml), glutamate salts (from 0.1 μg/ml to 5000.0 g/ml), mannitol (from 0.1 μg/ml to 5000.0 μg/ml), hydrogen phosphate salts (from 0.1 μg/ml to 5000.0 μg/ml), salts of dihydrophosphates (from 0.1 μg/ml to 5000.0 μg/ml), manganese salts (from 0.1 μg/ml to 5000.0 μg/ml),

In a In some embodiments invention, a method is provided in which to increase the growth rate, body weight gain, and other vital and commercially important characteristics of animals and aquaplankton, the feed is treated before feeding using the product M4 in an amount from 0.001 μg/ml to 10e5 μg/ml or compositions M4 and zinc salts (from 0.1 μg/ml to 5000.0 μg/ml) glycerin (from 0.1 μg/ml to 5000.0 μg/ml) sorbitol (from 0.1 μg/ml to 5000.0 μg/ml), boron salts (from 0.1 μg/ml to 5000.0 μg/ml), glutamate salts (from 0.1 μg/ml to 5000.0 μg/ml), mannitol (from 0.1 μg/ml to 5000.0 μg/ml), hydrogen phosphate salts (from 0.1 μg/ml to 5000.0 μg/ml), salts of dihydrophosphates (from 0.1 μg/ml to 5000.0 μg/ml) of manganese salts (from 0.1 μg/ml to 5000.0 μg/ml), followed by inactivation with carboxymethyl cellulose, sodium alginate in a ratio with the drug 0.5-1.0 to 100.0-1.0

In some embodiments invention, a method is provided in which to increase germination, growth rate, chlorophyll formation and yield when pelleting seeds for 3 seconds to 24 hours, treatment is carried out using the product M4 in an amount from 0.001 μg/ml to 10e5 μg/ml or compositions M4 and zinc salts (from 0.1 μg/ml to 5000.0 μg/ml) glycerin (from 0.1 μg/ml to 5000.0 μg/ml) sorbitol (from 0.1 μg/ml to 5000.0 μg/ml), boron salts (from 0.1 μg/ml to 5000.0 μg/ml), glutamate salts (from 0.1 μg/ml to 5000.0 μg/ml), mannitol (from 0.1 μg/ml to 5000.0 μg/ml), hydrogen phosphate salts (from 0.1 μg/ml to 5000.0 μg/ml), salts of dihydrophosphates (from 0.1 μg/ml to 5000.0 μg/ml), manganese salts (from 0.1 μg/ml to 5000.0 μg/ml) or reverse transcription inhibitors (from 0.1 μg/ml to 5000.0 μg/ml), integration/recombination (from 0.1 μg/ml to 5000.0 μg/ml), proteases (from 0.1 μg/ml to 5000.0 μg/ml), DNAse (from 0.1 μg/ml to 500.0 μg/ml), RNAse (from 0.1 μg/ml to 500.0 μg/ml), DNAse+RNAse (from 0.1 μg/ml to 500.0 μg/ml) and complexes of the listed drugs,

In some embodiments invention provides a method in which to increase germination, growth rate, chlorophyll formation and yield when pelleting seeds for 3 seconds to 24 hours, treatment is carried out using the product M4 in an amount from 0.001 μg/ml to 10e5 μg/ml or compositions M4 and zinc salts (from 0.1 μg/ml to 5000.0 μg/ml) glycerin (from 0.1 μg/ml to 5000.0 μg/ml) sorbitol (from 0.1 μg/ml to 5000.0 μg/ml), boron salts (from 0.1 μg/ml to 5000.0 μg/ml), glutamate salts (from 0.1 μg/ml to 5000.0 μg/ml), mannitol (from 0.1 μg/ml to 5000.0 μg/ml), hydrogen phosphate salts (from 0.1 μg/ml to 5000.0 μg/ml), salts of dihydrophosphates (from 0.1 μg/ml to 5000.0 μg/ml), manganese salts (from 0.1 μg/ml to 5000.0 μg/ml), or reverse transcription inhibitors (from 0.1 μg/ml to 5000.0 μg/ml), integration/recombination (from 0.1 μg/ml to 5000.0 μg/ml), proteases (from 0.1 μg/ml to 5000.0 μg/ml), DNAse (from 0.1 μg/ml to 500.0 μg/ml), RNAse (from 0.1 μg/ml to 500.0 μg/ml), DNAse+RNAse (from 0.1 μg/ml to 500.0 μg/ml) and complexes of the listed drugs with subsequent inactivation with carboxymethyl cellulose, sodium alginate in a ratio with the drug 0.5-1.0 to 100.0-1.0

In some embodiments invention, a method is provided in which to increase germination, growth intensity, chlorophyll formation and yield, seed dressing is performed for 1.0 minutes to 24 hours using the M4 product in an amount from 0.001 μg/ml to 10e5 μg/ml or compositions M4 and zinc salts (from 0.1 μg/ml to 5000.0 μg/ml) glycerol (from 0.1 μg/ml to 5000.0 μg/ml) sorbitol (from 0.1 μg/ml to 5000.0 μg/ml), boron salts (from 0.1 μg/ml to 5000.0 μg/ml), glutamate salts (from 0.1 μg/ml to 5000.0 μg/ml), mannitol (from 0.1 μg/ml to 5000.0 μg/ml), hydrogen phosphate salts (from 0.1 μg/ml to 5000.0 μg/ml), salts of dihydrophosphates (from 0.1 μg/ml to 5000.0 μg/ml) salts of manganese (from 0.1 μg/ml to 5000.0 μg/ml), or reverse transcription inhibitors (from 0.1 μg/ml to 5000.0 μg/ml), integration/recombination (from 0.1 μg/ml to 5000.0 μg/ml), proteases (from 0.1 μg/ml to 5000.0 μg/ml), DNAse (from 0.1 μg/ml to 500.0 μg/ml), RNAse (from 0.1 μg/ml up to 500.0 g/ml), DNAse+RNAse (from 0.1 μg/ml to 500.0 μg/ml) and complexes of the listed drugs

In some embodiments invention, a method is provided in which to increase germination, growth intensity, chlorophyll formation and yield, seed dressing is performed for 1.0 minutes to 24 hours using the M4 product in an amount from 0.001 μg/ml to 10e5 μg/ml or compositions M4 and zinc salts (from 0.1 μg/ml to 5000.0 μg/ml) glycerol (from 0.1 μg/ml to 5000.0 μg/ml) sorbitol (from 0.1 μg/ml to 5000.0 μg/ml), boron salts (from 0.1 μg/ml to 5000.0 μg/ml), glutamate salts (from 0.1 μg/ml to 5000.0 μg/ml), mannitol (from 0.1 μg/ml to 5000.0 μg/ml), hydrogen phosphate salts (from 0.1 μg/ml to 5000.0 μg/ml), salts of dihydrophosphates (from 0.1 μg/ml to 5000.0 μg/ml) salts of manganese (from 0.1 μg/ml to 5000.0 μg/ml), or reverse transcription inhibitors (from 0.1 μg/ml to 5000.0 μg/ml), integration/recombination (from 0.1 μg/ml to 5000.0 μg/ml), proteases (from 0.1 μg/ml to 5000.0 μg/ml), DNAse (from 0.1 μg/ml to 500.0 μg/ml), RNAse (from 0.1 μg/ml up to 500.0 μg/ml), DNAse+RNAse (from 0.1 μg/ml to 500.0 μg/ml) and complexes of the above drugs, followed by inactivation with carboxymethylcellulose, sodium alginate in a ratio with the drug 0.5-1.0 to 100.0-1.0

In some embodiments invention, a method is provided in which to increase germination, growth intensity, chlorophyll formation and yield, seed dressing is performed for 1.0 minutes to 24 hours using the M4 product in an amount from 0.001 μg/ml to 10e5 μg/ml or compositions M4 and zinc salts (from 0.1 μg/ml to 5000.0 μg/ml) glycerol (from 0.1 μg/ml to 5000.0 μg/ml) sorbitol (from 0.1 μg/ml to 5000.0 μg/ml), boron salts (from 0.1 μg/ml to 5000.0 μg/ml), glutamate salts (from 0.1 μg/ml to 5000.0 μg/ml), mannitol (from 0.1 μg/ml to 5000.0 μg/ml), hydrogen phosphate salts (from 0.1 μg/ml to 5000.0 μg/ml), salts of dihydrophosphates (from 0.1 μg/ml to 5000.0 μg/ml) salts of manganese (from 0.1 μg/ml to 5000.0 μg/ml), or reverse transcription inhibitors (from 0.1 μg/ml to 5000.0 μg/ml), integration/recombination (from 0.1 μg/ml to 5000.0 μg/ml), proteases (from 0.1 μg/ml to 5000.0 μg/ml), DNAse (from 0.1 μg/ml to 500.0 μg/ml), RNAse (from 0.1 μg/ml up to 500.0 μg/ml), DNAse+RNAse (from 0.1 μg/ml to 500.0 μg/ml) and complexes of the listed drugs, followed by planting in soil pretreated with the same drugs

In some embodiments invention, a method is provided in which for bioactivation of the prevention and treatment of bee diseases and increasing the amount of honey obtained, the hive is treated and fed with M4 products in an amount from 0.001 μg/ml to 10e5 μg/ml or compositions M4 and zinc salts (from 0.1 μg/ml to 5000.0 μg/ml) glycerol (from 0.1 μg/ml to 5000.0 μg/ml) sorbitol (from 0.1 μg/ml to 5000.0 μg/ml), boron salts (from 0.1 μg/ml to 5000.0 μg/ml), salts glutamate (from 0.1 μg/ml to 5000.0 μg/ml), mannitol (from 0.1 μg/ml to 5000.0 μg/ml), hydrogen phosphate salts (from 0.1 μg/ml to 5000.0 μg/ml), dihydrophosphate salts (from 0.1 μg/ml to 5000.0 μg/ml) manganese salts (from 0.1 μg/ml to 5000.0 μg/ml), or reverse transcription inhibitors (from 0.1 μg/ml to 5000.0 μg/ml), integration/recombination (from 0.1 μg/ml to 5000.0 μg/ml), proteases (from 0.1 μg/ml to 5000.0 μg/ml), DNAse (from 0.1 μg/ml to 500.0 μg/ml), RNAse (from 0.1 μg/ml to 500.0 μg/ml), DNAse+RNAse (from 0.1 μg/ml to 500.0 μg/ml) and complexes of pere studied drugs

In some embodiments invention provides a method for correcting the condition of the skin and/or subcutaneous tissue of animals and humans in various diseases, including seborrheic dermatitis, neuroderma, Psoriasis, Herpes, papillomatosis, mycoses, erysipelas, trophic and diabetic ulcers, trauma, acne bedsores, folliculitis, furunculosis, angulitis (jam) alopecia using M4 product in an amount from 0.001 μg/ml to 10e5 μg/ml or compositions M4 and zinc salts (from 0.1 μg/ml to 5000.0 μg/ml) glycerol (from 0.1 μg/ml to 5000.0 g/ml) sorbitol (from 0.1 μg/ml to μg/ml), mannitol (from 0.1 μg/ml to 5000.0 μg/ml), salts of hydrogen phosphates (from 0.1 μg/ml to 5000.0 μg/ml), salts of dihydrophosphates (from 0.1 μg/ml to 5000.0 μg/ml), manganese salts (from 0.1 μg/ml to 5000.0 μg/ml), hyaluronic acid salts or reverse transcription inhibitors (from 0.1 μg/ml to 5000.0 μg/ml), integration/recombination (from 0.1 μg/ml to 5000.0 μg/ml), proteases (from 0.1 μg/ml to 5000.0 μg/ml), DNAse (from 0.1 μg/ml to 500.0 μg/ml), RNA zy (from 0.1 μg/ml to 500.0 μg/ml), DNAse+RNAse (from 0.1 μg/ml to 500.0 μg/ml) and complexes of the above drugs, while the drugs are gels and/or emulsions and/or ointments and/or liquid

In some embodiments invention provides a method for correcting the state of the mucous membranes of the sinuses and/or the mucous membranes of the urinary bladder, in which using the drug M4 in an amount from 0.001 μg/ml to 10e5 μg/ml or compositions M4 and zinc salts (from 0.1 μg/ml to 5000.0 μg/ml) glycerol (from 0.1 μg/ml to 5000.0 μg/ml) sorbitol (from 0.1 μg/ml to μg/ml), mannitol (from 0.1 μg/ml to 5000.0 μg/ml), hydrogen phosphate salts (from 0.1 μg/ml to 5000.0 μg/ml), dihydrophosphate salts (from 0.1 μg/ml to 5000.0 μg/ml), manganese salts (from 0.1 μg/ml to 5000.0 μg/ml), hyaluronic acid salts or reverse transcription inhibitors (from 0.1 μg/ml to 5000.0 μg/ml), integration/recombination (from 0.1 μg/ml to 5000.0 μg/ml), proteases (from 0.1 μg/ml to 5000.0 μg/ml), DNAse (from 0.1 μg/ml to 500.0 μg/ml), RNAse (from 0.1 μg/ml to 500.0 μg/ml), DNAse+RNAse (from 0.1 μg/ml to 500.0 μg/ml) and complexes of the above drugs, while the drugs are gels and/or emulsions and/or liquids, in which the drug is injected into the treatment cavity and is either removed after rinsing or inactivated or remains in the cavity

In some embodiments invention provides a method for correcting the state of the oral cavity, including periodontal and endodontic mucosa, as well as cysts and granulomas, using the M4 product in an amount from 0.001 μg/ml to 10e5 μg/ml or compositions M4 and zinc salts (from 0.1 μg/ml to 5000.0 μg/ml) glycerol (from 0.1 μg/ml to 5000.0 μg/ml) sorbitol (from 0.1 μg/ml to μg/ml), mannitol (from 0.1 μg/ml to 5000.0 μg/ml), hydrogen phosphate salts (from 0.1 μg/ml to 5000.0 μg/ml), salts of dihydrophosphates (from 0.1 μg/ml to 5000.0 μg/ml), manganese salts (from 0.1 μg/ml to 5000.0 μg/ml), hyaluronic acid salts or reverse transcription inhibitors (from 0.1 μg/ml to 5000.0 μg/ml), integration/recombination (from 0.1 μg/ml to 5000.0 g/ml), proteases (from 0.1 μg/ml to 5000.0 μg/ml), DNAse (from 0.1 μg/ml to 500.0 μg/ml), RNAse (from 0.1 μg/ml to 500.0 μg/ml), DNAse+RNAse (from 0.1 μg/ml to 500.0 μg/ml) and complexes of the above drugs, while the drugs are gels and/or emulsions and/or liquids,

In some embodiments invention provides a correction method for correcting the condition of the eyes, including conjunctivitis and dacryocystitis, using M4 product in an amount from 0.001 μg/ml to 10e5 μg/ml or compositions M4 and zinc salts (from 0.1 μg/ml to 5000.0 μg/ml) glycerol (from 0.1 μg/ml to 5000.0 μg/ml) sorbitol (from 0.1 μg/ml to μg/ml), mannitol (from 0.1 μg/ml to 5000.0 μg/ml), hydrogen phosphate salts (from 0.1 μg/ml to 5000.0 μg/ml), salts of dihydrophosphates (from 0.1 μg/ml to 5000.0 μg/ml), manganese salts (from 0.1 μg/ml to 5000.0 μg/ml), hyaluronic acid salts or reverse transcription inhibitors (from 0.1 μg/ml to 5000.0 μg/ml), integration/recombination (from 0.1 μg/ml to 5000.0 μg/ml), proteases (from 0.1 μg/ml to 5000.0 μg/ml), DNAse (from 0.1 μg/ml to 500.0 μg/ml), RNAse (from 0.1 μg/ml to 500.0 μg/ml), DNAse+RNAse (from 0.1 μg/ml to 500.0 μg/ml) and complexes of the above drugs, while the drugs are gels and/or emulsions and/or liquids.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows bioactive effect of M421 and M451 on plant growth of (A) wheat (B) Oryza sativa. (C) Cucurbita pepo. (D) Cucumis sativus, (E) Nicotiana rustica, (G) Glycine hispida.

FIG. 2 shows an example of the 6 day growth of a control culture and in the presence of M421

FIG. 3 shows the effects of compounds of M4 group on seed dressing

FIG. 4 shows the result of 12 days treatment

FIG. 5 shows the effects of M4 group of compounds on the treatment of bees infected with P. larvae. (A) Untreated, (B) Treated. Honeycombs with dead bees are marked in red Honeycombs with honey, not covered with wax are shown in blue.

FIG. 6 shows the growth pattern with different types of processing

FIG. 7 shows the effects of different compounds on fish eggs processing

FIG. 8 shows microbial growth from branchial arches. (A) Before treatment, (B) After treatment.

FIG. 9 shows the effects of tested compounds on water processing. (A) Before treatment, (B) After treatment.

FIG. 10 shows the effect of tested compounds on feed processing. (A) Before treatment, (B) After treatment.

FIG. 11 shows fish the results of weekly treatment with (M4 0.1%). (A) Before treatment, (B) After treatment.

FIG. 12 shows clinical representation of a clinical case. (A) Before using M491, (B) After using M491.

FIG. 13 shows the effects of tested compounds on seborrheic dermatitis. (A) Before treatment, (B) After 7 days of treatment.

FIG. 14 shows the diabetic ulcer treatment results with M421. A) Before treatment, (B) After 5 weeks of treatment.

FIG. 15 shows the use of compounds of M4 group for the treatment of psoriasis treatment (A) Before treatment, (B) After 7 days of treatment.

FIG. 16 shows the use of M421 for the treatment of contact dermatitis. (A) Before treatment, (B) After 7 days of treatment.

FIG. 17 shows the use of M421 for the treatment of eczema. (A) Before treatment, (B) After treatment.

FIG. 18 shows the use of M491 for the treatment of Herpes zoster (A) Before treatment, (B) After treatment.

FIG. 19. The use of M421 for the treatment of folliculitis (A) Before treatment, (B) After 7 days of treatment.

FIG. 20 shows the use of M491 for the treatment of epidermophytosis (A) Before treatment, (B) After treatment.

FIG. 21 shows the use of M491 for the treatment of complicate caries (A) Before treatment, (B) After treatment.

FIG. 22 shows washout from the affected hoof before and after treatment of hoof rot (A) Before treatment, (B) After treatment.

FIG. 23 shows effect of M451 on seeds growth in soils with high salinity.

### DETAILED DESCRIPTION OF THE INVENTION

The present invention relates to products and methods of their use for controlling living organisms for their bioactivity, improving their habitat in order to increase the biological activity of living beings in various conditions, as well as preventing and treating diseases of plants, animals and humans. Biologically active products that are used to achieve these goals include M4 (Poly-N1-hydrazino (imino) methyl-1,6-hexanediamine—compositions of the product M4 and zinc salts, glycerin, sorbitol, boron salts, glutamate, mannitol, Sodium hydrogenphosphate, Sodium dihydrogen phosphate, manganese salts, glutamic acid, hyaluronic acid, reverse transcription inhibitors, integration/recombination inhibitors, protease inhibitors, DNAses, RNAses, DNAse+RNAse complexes.

### Definitions

Aquaculture—cultivated, including by means of artificial breeding and rearing, aquatic biological resources (fish, aquatic animals and plants and their hybrid forms);
Water, ponds, artificial containers with flowing and un-flowing water, aquariums for shops, aquariums for breeding and maintenance of decorative fishes
Reverse transcription inhibitors (Nevirapine, Penciclovir, Tenofovir disoproxil, Zidovudine, Foscarnet, Efavirenz, Stavudine, Delavirdine, Lamivudine, Adefovir dipivoxil Etravirine Abacavir)
Integration/recombination inhibitors (Dolutegravir Elvitegravir Libertsin Raltegravir)
Protease inhibitors (BOCEPREVIR TELAPREVIR, SIMEPREVIR, ASUNAPREVIR Lopinavir+ritonavir)
Product M4 Poly-N1-hydrazino (imino) methyl-1,6-hexanediamine
Fertigation (application of liquid fertilizers simultaneously with watering),
Zooplankton (fish, crustaceans and molluscs)

| | Composition of |
|---|---|
| Products | M4 group of products |
| Control | — |
| M4 glycerol 0.1 μg/ml | M4 * |
| M411 | M4 + glycerol 0.1 μg/ml |
| M412 | M4 + glycerol 0.01 μg/ml |
| M413 | M4 + glycerol 0.001 μg/ml |
| M414 | M4 + glycerol 0.0001 μg/ml |
| ZnSO4 0.01% | ZnSO4 0.01% |
| M421 | M4 + ZnSO4 0.01% |
| M422-1 Sorbitol 0.1 μg/ml | M4 + ZnSO4 0.01% + glycerol 0.1 μg/ml |
| M431 | M4 + sorbitol 0.1 μg/ml |
| M432 | M4 + sorbitol 0.01 μg/ml |
| M433 | M4 + sorbitol 0.001 μg/ml |
| M434 | M4 + sorbitol 0.0001 μg/ml |
| M434-2 | M4 + ZnSO4 0.01% + sorbitol 0.01 μg/ml |
| MnC12 (0.01%) | MnC12 (0.01%) |
| M441 | M4 + MnC12 (0.01%) |
| NaH2PO4 (0.01%) | NaH2PO4 (0.01%) |
| M451 | M4 + NaH2PO4 (0.01%) |
| Na2HPO4 (0.01%) | Na2HPO4 (0.01%) |
| M452 | M4 + Na2HPO4 (0.01%) |
| Na₂B₄O₇ (0.01%) | Na₂B₄O₇ (0.01%) |
| M461 | M4 + Na₂B₄O₇ (0.01%) |
| Monosodium | Monosodium glutamate (0.01%) |
| glutamate (0.01%) | |
| M471 | M4 + monosodium glutamate (0.01%) |
| Mannitol (0.01%) | Mannitol (0.01%) |
| M481 | M4 + mannitol (0.01%) |
| Sodium | Sodium hyaluronate (0.1%) |
| hyaluronate (0.1%) | |
| M491 | M4 + Sodium hyaluronate (0.1%) |

### EXAMPLES

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

### Example 1. Bioactive Effect of M4 Group of Products

Wheat seeds (Triticum aestivum L., healthy, not infected) were washed with water with soap, then without soap. Then surface sterilization with 70% ethyl alcohol was carried out for 2-3 minutes. Then the seeds (8 pieces) were laid out on potato dextrose agar (https://himedialabs.com/TD/M096.pdf).

Treatment with test products. The seeds were soaked in nuclease solutions at 37° C. for 1 hour (100 μg/ml), in a solution of M421, M422-1, M431, M432, M433, M434, M434-2, M441, M451, M452, M461, M471, M481, M491 0.5% for 1 hour Together: 1 hour at 37° C. Data are presented in table 1.

TABLE 1
Bioactive effect of M4 group of products

| | Germination (%) | Median shoot |
|---|---|---|
| | at day 5 | length (mm) |
| Products | Day 3 | Day 3 |
| Control | 54 | 15.5 |
| M4 (0.5%) | 73 | 21.6 |
| DNAse | 75 | 24.6 |
| DNAse + M4 (0.5%) | 74 | 22.7 |
| Chlorhexidine (0.5%) | 54 | 14.6 |
| DNAse + | 76 | 24.1 |
| Chlorhexidine (0.5%) | | |
| PHMG** (0.5%) | 51 | 17.2 |
| DNAse + PHMG | 73 | 19.4 |
| M421 | 96 | 22.7 |
| M461 | 98 | 21.6 |
| M471 | 98 | 27.8 |
| M481 | 97 | 23.1 |
| M422-1 | 92 | 22.2 |
| M431 | 98 | 21.1 |
| M433 | 94 | 25.5 |
| M434 | 91 | 25.2 |
| M434-2 | 90 | 23.4 |
| M441 | 98 | 24.3 |
| M432 | 96 | 22.7 |
| M451 | 94 | 24.3 |
| M452 | 95 | 21.7 |
| M491 | 98 | 23.8 |

PHMG** Poly(hexamethyleneguanidine hydrochloride, (C₇H₁₆N₃Cl)ₙ,

Products of the M4 group and DNase increase germination and the shoot size. The bioactive effect on seed germination of the tested products of the M4 group exceeded that of M4. These data indicate that products M4, M421, M421, M461, M471 and M481, acting on seeds (seeds that have undergone preliminary antimicrobial treatment, according to existing guidelines), have a previously unknown bioactivity effect, which is not associated with their antimicrobial activity. The bioactivity effect is more pronounced in the compositions, associated with the presence of DNA receptors on seeds. With the simultaneous action of nucleases and products of the M4 group, the effect of action of both DNase and M4 disappears. Nothing of the kind is observed when using the antiseptic products Chlorhexidine and PHMG, which indicates that they have no bioactive effect.

### Example 2. Plant Growth Management Under Stress Conditions Using Inhibitors of Integration, DNase, RNase and M4 Group of Products

Wheat seeds (Triticum aestivum L., healthy, not infected) were soaked for 1 hour at 37° C. grains in product solutions: 100 μg/ml.

Next, the seeds were planted in sterile soil, at a height of 5 cm from the bottom of the pot, covered by 1 cm of soil. The cultivation temperature was 18° C., instead of the required 23° C. The assessment of the state of the plants was carried out after 5 days (table 2).

TABLE 2
Bioactive effect of M4 group of products
under the stress conditions

| | Shoot | Root | |
|---|---|---|---|
| | length | length | Emergence |
| Product | (cm) | (cm) | at day 5 |
| Control | 6.5 | 1.7 | 47% |
| Raltegravir | 9.8 | 2.6 | 71% |
| Elvitegravir | 9.9 | 2.6 | 65% |
| DNAse | 6.9 | 2.1 | 64% |
| M4 (0.1%) | 8.9 | 2.9 | 73% |
| Raltegravir + DNAse | 10.4 | 3.1 | 73% |
| Raltegravir + M4 | 3.2 | 1.3 | 40% |
| DNAse + M4 | 7.9 | 2.7 | 43% |
| Raltegravir + DNAse + | 8.2 | 3.5 | 93% |
| M4 | | | |
| Raltegravir + DNAse + | 8.4 | 2.4 | 44% |
| RNase + M4 | | | |
| Raltegravir + DNAse + | 4.4 | 1.3 | 49% |
| RNAse + M4 | | | |
| Chlorhexidine (05%) | 6.3 | 1.9 | 53% |
| DNAse + Chlorhexidine (0.5%) | 6.4 | 1.8 | 64% |
| Raltegravir + Chlorhexidine (0.5%) | 9.6 | 2.2 | 62% |
| PHMG (05%) | 7.6 | 2.3 | 48% |
| Raltegravir + PHMG (05%) | 9.7 | 2.5 | 59% |

The data obtained indicate that integrase and M4 inhibitors act as bioactive compounds of plant growth and, at the same time, increase plants' stress resistance. Integrase and DNase inhibitors have a synergistic effect of action. The M4 acts as an inhibitor of integrase activity, while itself acting as a bioactive compound. The latter indicates that DNA on the surface of seed cells is associated with the main target for M4, and the implementation of the action of an integrase inhibitors require the presence of a target on the cells, which is blocked by M4. Comparative products under these conditions did not show a bioactive effect.

### Example 3. Regulation of Plant Development by the Claimed Products

Plant: wheat. Treatment: The wheat seeds were treated with various nucleases or products of the M4 group for 1 hour, at 37° C. Next, the grain was planted in a sterile soil, at a height of 5 cm from the bottom of the pot, covered by 1 cm of soil. Cultivation temperature 23° C. Data are shown in table 3.

TABLE 3
Regulation of plant development by the claimed products

| Samples | | Root | Shoot | | Leaf | Leaf | | |
|---|---|---|---|---|---|---|---|---|
| treated | Emergence | length, | length | Weight | length | width | Number | Chlorophyll |
| with | (%) | (cm) | (cm) | (g) | (cm) | (cm) | of leaves | total |
| Untreated | 33 | 12.5 | 5.5 | 0.6 | 2.6 | 1.7 | 2 + 1 | 47.6 |
| seeds | | | | | | | | |
| DNAse | 67 | 15.2 | 5.8 | 0.6 | 2.8 | 1.4 | 2 + 1 | 49.1 |
| RNAse | 50 | 11.0 | 6.0 | 0.5 | 2.4 | 1.4 | 2 + 1 | 41.1 |
| DNAse + | 50 | 11.2 | 6.5 | 0.6 | 2.7 | 1.5 | 2 + 1 | 45.4 |
| RNAse | | | | | | | | |
| M4 | 50 | 15.9 | 6.2 | 0.7 | 2.7 | 1.5 | 2 + 1 | 49.6 |
| M421 | 54 | 15.9 | 6.2 | 0.7 | 2.8 | 1.8 | 2 + 1 | 49.8 |
| M481 | 53 | 15.8 | 6.1 | 0.7 | 2.7 | 1.7 | 2 + 1 | 49.7 |
| M461 | 52 | 15.8 | 6.2 | 0.7 | 2.8 | 1.7 | 2 + 1 | 49.7 |

The data obtained indicate that the treatment with the tested products changes the parameters of plant growth. DNase, as well as products M4, M421, M461, and M481, have the greatest bioactive effect.

### Example 4. Bioactivity of Reverse Transcription, Integration and Protease Inhibitors

Plant: wheat

Processing: The seeds were treated with various products for 1 hour, at 37° C.

Next, the grain is planted in sterile soil, at a height of 5 cm from the bottom of the pot, covered by 1 cm of soil. Cultivation temperature 23° C. Data are shown in table 4.

TABLE 4
Bioactivity of reverse transcription, integration
and protease inhibitors on plants' growth

| | | Shoot | Root | Chlorophyll |
|---|---|---|---|---|
| | Emergence | length | lenght | total |
| Product | (%) | (median) | (median) | (mg/g) |
| Control | 35 | 0 | 0 | 45.6 |
| Nevirapine | 25 | 0 | 0 | 52.9 |
| Etravirine | 55 | 13 | 9 | 58.7 |
| Tenofovir | 45 | 14 | 9 | 57.7 |
| Lamivudine | 55 | 13 | 9 | 54.3 |
| Abacavir | 50 | 13 | 11 | 51.8 |
| Azidothymidine | 65 | 15 | 10 | 49.9 |
| Libercin | 25 | 0 | 0 | 57.0 |
| Raltegravir | 60 | 12 | 8 | 56.9 |
| VTL | 50 | 17 | 12 | 56.8 |
| Lopinavir + | 75 | 16 | 12 | 53.8 |
| ritonavir | | | | |

The products used affect the development of plants. The products have a bioactive effect in relation to the various properties of plants.

### Example 5. Bioactivity of Complexes of Reverse Transcription, Integration and Protease Inhibitors

Plant: wheat

Treatment: The seeds were treated with various products for 1 hour, at 37° C.

Next, the seeds were planted in sterile soil, at a height of 5 cm from the bottom of the pot, covered by 1 cm of soil. Cultivation temperature 23° C. Data are shown in table 5.

TABLE 5
Bioactivity of complexes of reverse transcription, integration
and protease inhibitors on plants growth.

| | Emergence | Shoot | Root | Chlorophyll |
|---|---|---|---|---|
| Product | (%) | length | lenght | total (mg/g) |
| Control | 33 | 3.2 | 1.0 | 23.6 |
| Etravirine | 75 | 7.4 | 5.5 | 25.0 |
| Raltegravir | 42 | 5.7 | 1.9 | 25.9 |
| Lopinavir | 80 | 6.5 | 3.5 | 27.8 |
| ritonavir | | | | |
| DNase | 73 | 5.2 | 3.6 | 24.7 |
| RNase | 46 | 3.8 | 1.7 | 35.5 |
| Etravirine + | 67 | 7.4 | 6.1 | 28.9 |
| DNase | | | | |
| Etravirine + | 54 | 7.5 | 4.5 | 33.6 |
| RNase | | | | |
| Raltegravir + | 83 | 7.3 | 9.3 | 32.9 |
| DNase | | | | |
| RNase + | 79 | 7.0 | 8.5 | 28.4 |
| Raltegravir | | | | |
| DNase + | 74 | 4.8 | 7.7 | 28.2 |
| lopinavir | | | | |
| ritonavir | | | | |
| RNase + | 71 | 4.4 | 7.2 | 34.9 |
| lopinavir | | | | |
| ritonavir | | | | |
| Trypsin | 49 | 2.5 | 5.8 | 30.3 |
| Proteinase K | 42 | 0.0 | 4.3 | 35.2 |

The results indicate a pronounced bioactive effect of the tested complex products on plant development. At the same time, germination can be increased by 250%, chlorophyll content by 50%, root length by 800%, and shoot by 150%,

### Example 6. Bioactive Effect of M421 on Plants Growth

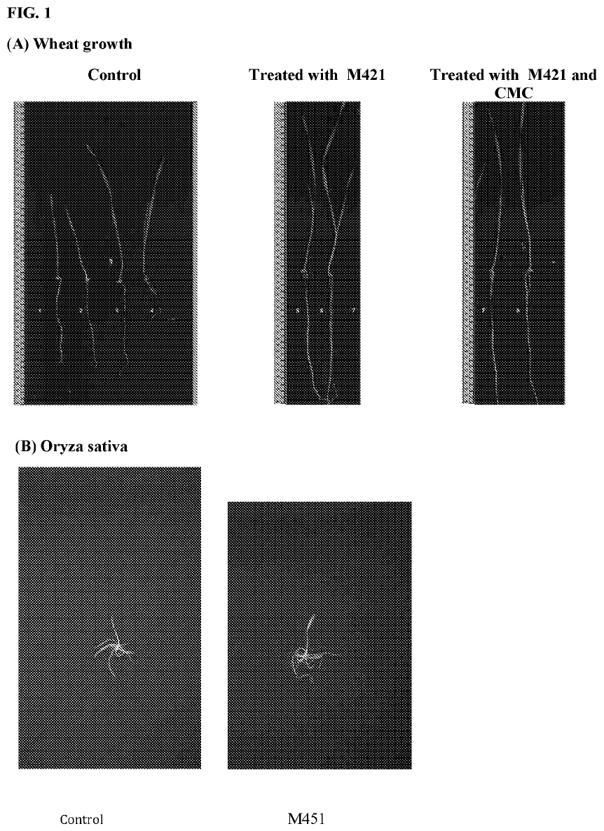

A healthy grain of (FIG. 1A) wheat is placed in the soil 6 cm from the bottom of a soil-filled pot, on top of 1 cm of soil. Cultivation temperature 23° C. GROWTH MODE: 7 days. IRRIGATION MODES: (#1) distilled water, (#2) 1st day M421 (0.5%), after an hour watering—carboxymethyl cellulose (CMC) (0.5%), 3-5-7 days—distilled water, (#3) 1st day M421 (0.5%), 3-5-7 days—distilled water. Data are shown in FIG. 1A.

The bioactive effect of M421 was manifested in an increase in the length of shoots in treated seeds up to 19 mm compared to 14 mm in untreated seeds and a root length up to 19.1 mm compared to 12.7 in the control group.

We also found that M451 0.1% increased the growth of (FIG. 1B) Oryza sativa, (FIG. 1C) Cucurbita pepo, (FIG. 1D) Cucumis sativus, (FIG. 1E) Nicotiana rustica, (FIG. 1G) Glycine hispida.

These data clearly show the acceleration of plants growth following M451 treatment.

### Example 7. Differentiation of Bioactivity, Antimicrobial Activity and Function of Fertilizer (Nitrogen Source) for Plants in the Claimed Products

Seed—Wheat

Treatment mode: watering with products (1000 μg/ml, 200 ml for 20 grains) one-time, then watering with plain water. Untreated seeds were planted in sterile soil, at a height of 5 cm from the bottom of the pot, covered by 1 cm of soil. Cultivation temperature 23° C.

Nitrogen content in the products used:
Chlorhexidine—27.7%
M4 and its complexes—32.1%
PHMG—32.0%
Ammonium nitrate—35%

The results were assessed on day 5 (Table 6).

TABLE 6
Bioactive effects of M4 group of the compounds

| | Emergence | Shoot | Root |
|---|---|---|---|
| Product | (%) | length | length |
| Control | 45 | 6.0 | 6.0 |
| Ammonium nitrate | 50 | 7.0 | 4.2 |
| Chlorhexidine | 50 | 6.9 | 3.6 |
| Chlorhexidine (0.5%) + | 50 | 7.3 | 5.7 |
| Ammonium Nitrate | | | |
| PHMG | 45 | 5.6 | 6.1 |
| PHMG (0.5%) + | 45 | 5.5 | 3.6 |
| Ammonium nitrate | | | |
| M4 | 60 | 8.5 | 8.0 |
| M4 + Ammonium nitrate | 65 | 8.5 | 5.8 |
| M411 | 60 | 8.3 | 8.2 |
| M421 | 68 | 8.6 | 8.0 |
| M434-1 | 65 | 8.5 | 8.0 |
| M451 | 60 | 8.2 | 8.3 |
| M452 | 65 | 8.5 | 8.0 |
| M481 | 65 | 8.7 | 8.3 |

The data obtained indicate that the comparison products, which have antimicrobial activity and have a similar amount of nitrogen in the molecule, as well as nitrogen-containing fertilizer, do not have a stimulating effect similar to what is recorded under the action of the product M4 and its compositions. The addition of ammonium nitrate to the reference products and the M4 product did not lead to a significant change in plant growth. Thus, the data obtained indicate that the claimed products have a bioactive effect that is not associated either with their antimicrobial activity, or with the presence of nitrogen molecules in their composition, which can be used as a fertilizer.

### Example 8. Effect of M4 Products on Plant Pathogens

The effect of the M4 group of products was tested was tested on 80 strains of fungi of the genus Fusarium sp. (F. culmorum, F. graminearum, F. sporotrichioides, F. oxysporum, F. solani) obtained from collections of various countries.

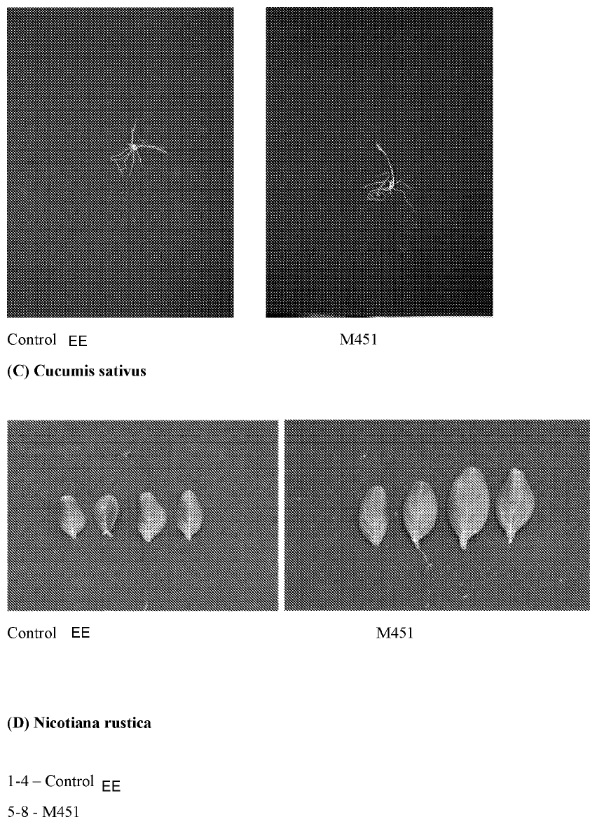

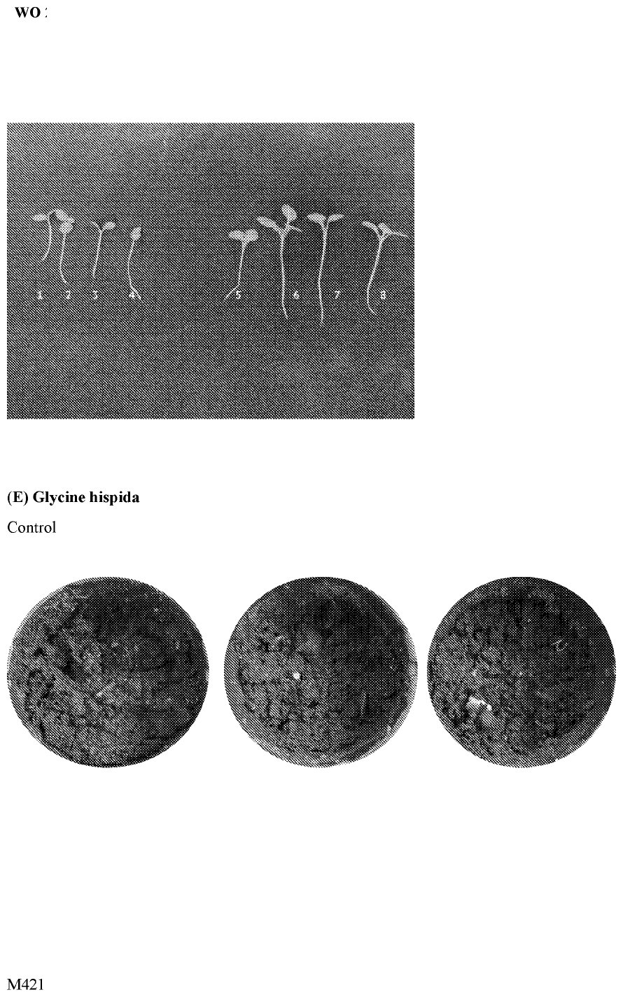

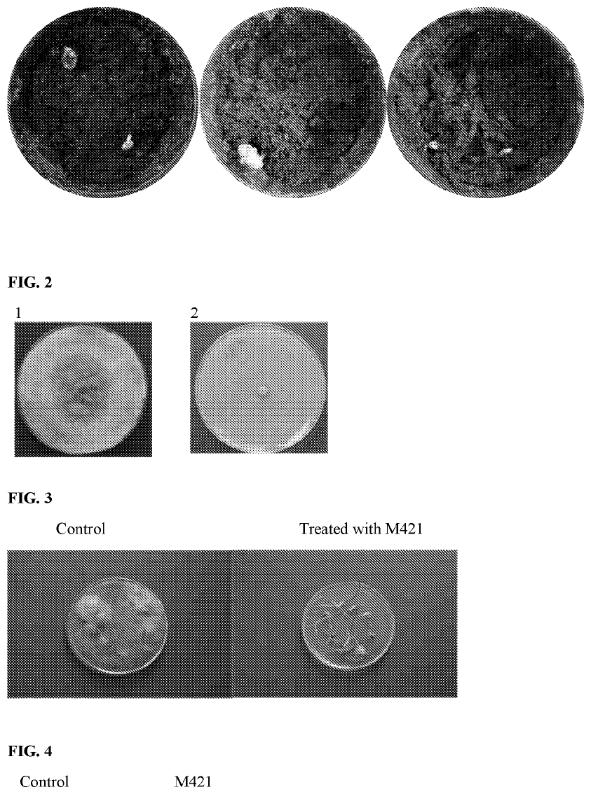

The fungi were grown on potato-dextrose agar. The agar (3 mm in diameter) from the zone of active fungal growth was cut and transferred into petri dish filled with the fresh solid medium supplemented with tested compounds, that inside the nutrient agar had a hole of 3 mm. The agar circle with fungi was transferred to this new Petri dish and placed inside this 3 mm hole. The presence and intensity of further growth of fungi was controlled. Data are shown in table 7 and FIG. 2.

TABLE 7
Effect of M4 products on plant pathogens

| | The number of strains that gave the | | | | |
|---|---|---|---|---|---|
| | minimum growth on day 6 (%) | | | | |
| Product | Control 0% | 0.5% | 0.1% | 0.2% | 0.05% |
| Control | 100 | — | — | — | — |
| M4 | — | 9 | 35 | 44 | 80 |
| M421 | — | 7 | 33 | 42 | 77 |
| M434-2 | — | 7 | 30 | 41 | 75 |
| M461 | — | 8 | 33 | 42 | 78 |
| M481 | — | 8 | 33 | 41 | 75 |

These data clearly show that tested compounds M421, M434-2, M461, M481 possess higher antimicrobial activity compared with M4. This fact is surprising, since the concentrating of M4 in these compounds is identical and excipients used, themselves do not possess any antimicrobial activity.

The tested products showed high activity against fungi of the genus Fusarium.

### Example 9. Use of Compounds from M4 Group to Treat Soil

Seeds: Wheat. (1) healthy seeds and (2) “infected seeds, initially infected with fungi of Fusarium genus. Soil: sterile (treated with 120° C., 1 atm, 40 minutes). Healthy grain was introduced into the soil at a distance of 6 cm from the bottom of a pot filled with earth, covered by 1 cm of soil. Growth regimen 7 days. Watering 1-3-5-7 days. Groups:

1. “healthy seeds”: watering on days 1-3-5-7 with distilled water.

2. “healthy seeds”: watering on day 1 with products of M4 group or their excipients, watering on days 3-5-7 with distilled water.

3. “infected seeds”: watering on days 1-3-5-7 days with distilled water.

4. “infected seeds”: watering on day 1 with products of M4 group or their excipients, watering on days 3-5-7 with distilled water.

Data are shown in table 8.

TABLE 8
Plant growth after a single watering with the product

| | Seedling length | Root length |
|---|---|---|
| Group | (cm) | (cm) |
| Healthy seed, control | 8.2 | 7.5 |
| Healthy seed, M4 | 10.8 | 9.6 |
| Healthy seed, ZnSO4 0.01% | 8.0 | 7.1 |
| Healthy seed, M421 | 11.3 | 9.8 |
| Healthy seed, Na₂HPO4 (0.01%) | 8.4 | 7.7 |
| Healthy seed, M461 | 15.3 | 11.5 |
| Infected seed, control | 7.6 | 6.6 |
| Infected seed, M4 | 10.4 | 9.3 |
| Infected seed, ZnSO4 0.01% | 6.1 | 5.7 |
| Infected seed, M421 | 12.2 | 10.5 |
| Infected seed, Na₂HPO4 (0.01%) | 6.1 | 5.9 |
| Infected seed, M461 | 14.4 | 10.6 |

The results obtained indicate that a single watering of the soil with the claimed products have bioactive effect on the growth of plants in sterile soil. Bioactive effect is more prominent for the infected seed. At the same time the bioactive effect is not realized due to the antimicrobial activity, since although M4 possesses similar antimicrobial activity to M421 and M461, its effects on plants growth is less.

### Example 10. Seed Dressing

Seeds: conditionally healthy wheat. Soil: sterilized at 120° C., 1 atm, 40 minutes.

Culture medium—potato dextrose agar (https://himedialabs.com/TD/M096.pdf).

1. Control. The seeds are soaked in saline for 3 hours, next placed on a nutrient medium with subsequent 7 days of growth.

2. Etchant M421 0.1% seeds are soaked in the solution for 3 hours, next placed on a nutrient medium with subsequent 7 days of growth.

Data are shown in FIG. 3.

### Example 11. Bioactive Effect of Products on the Vegetative in Some Embodiments Plant

Plant: Large-fruited fodder pumpkin

Application method—Spraying

M421 at 0.5% concentration was sprayed on the leaf surface at a dosage of 200 μg/ml 1 time in 3 days, 0.3 ml/cm2, for 12 days. Control group was treated with water.

The experiment was set up according to the methodology described in https://www.ncbi.nlm.nih.gov/pmc/articles/PMC5855050/. Data are presented in FIG. 4 and table 9.

TABLE 9
Effects of the tested compounds on Chlorophyll content

| | Control | M421 |
|---|---|---|
| Chlorophyll a | 31 mg/g | 48 mg/g |
| Chlorophyll b | 18 mg/g | 29 mg/g |
| Chlorophyll, total | 49 mg/g | 77 mg/g |

Chlorophyll a: A special form of chlorophyll used for oxygenic photosynthesis. It absorbs light most strongly in the violet-blue and orange-red parts of the spectrum.

Data indicate that the product M421 stimulates an increase in the amount of chlorophyll in the leaves by 50% (FIG. 4).

### Example 12. Bioregulation of the Full Cycle of Plant Growth

### Red Turkish Carnation Seeds

Seeds from one package were placed in the individual microtubes (1.5 ml) with added solutions of:
1) 1 ml sterile distilled H2O.
2) 1 ml DNase solution (100 μg/ml) in sterile distilled H2O
3) 1 ml RNase solution (100 μg/ml) in sterile distilled H2O
4) 1 ml DNase+RNase solutions (100 μg/ml each) in sterile distilled H2O

The tubes were incubated in a ventilated incubator at 37° C. for 60 minutes.

After incubation, the solutions were removed and the seeds were washed with 1 ml of sterile H2O at room temperature, stirring with shaking. Sowing was carried out in seedling peat pots filled with soil. Germination in a greenhouse made of dense polyethylene. Watering 1 time in 2 days with room temperature tap water. Lighting from 7 am to 20 μm with LED light Uniel, 16 W for plants. Results are presented in table 10.

TABLE 10
Bioregulation of the full cycle of plant growth

| | Sign | | | | | | | |
|---|---|---|---|---|---|---|---|---|
| | | | | | | | Weight of | |
| | The | | | | | | seeds | Tolerance |
| | appearance | | Height | | The | The | on | to |
| | of the | | on | Root | emergence | beginning | day | stress |
| | first | Emergence | day | length at | of the | of | 258/12 | (Drying |
| Probe | leaves | % | 99 | 99 days | buds | flowering | plants (g) | test) |
| Control | 6th day | 95 | 5+/−1 | 5+/−0.5, | 205 day | 212 day | 0.001 | Irreversible |
| | | | | slightly | | | | drying of |
| | | | | branched | | | | the lower |
| | | | | | | | | leaves |
| DNase | 5th day | 95 | 7+/−1 | 7+/−0.5 | 174 day | 183 day | 0.24 | Reversible |
| | | | | branched | | | | drying |
| RNase | 4th day | 84 | 6+/−1 | 4+/−0.5, | 177 day | 186 day | 0.25 | The |
| | | | | moderately | | | | leaves |
| | | | | branched | | | | did not |
| | | | | | | | | change |
| DNase | 5th day | 96 | 4+/−1 | 8+/−0.5, | 172 day | 178 day | 0.17 | Reversible |
| + | | | | broad | | | | drying |
| RNase | | | | base, | | | | |
| | | | | extremely | | | | |
| | | | | branched | | | | |

The data obtained indicate that treatment with nucleases affects the entire cycle of plant development. Removal of RNA receptors with RNase during seed treatment increases the rate of appearance of the first leaves, the appearance of buds, the beginning of flowering, increases stress tolerance and the maximum mass of seeds. Removal of DNA receptors increases germination, increases the growth rate of the root and its branching, the rate of bud appearance, the beginning of flowering, increases stress resistance and maximum seed weight.

Removal of DNA and RNA receptors increases germination, growth rate, rate of appearance of the first leaves, root growth and branching, emergence of buds, the beginning of flowering, increases stress resistance and maximum seed weight

Thus, the bioactive effect of seed treatment with nucleases was registered for all parameters of plant growth.

### Example 13. Activity of M4 Group of Products was Assessed Against Paenibacillus larvae

Activity of M4 group of products was assessed against Paenibacillus larvae, that is a highly virulent disease afflicting honey bees. The minimum inhibitory concentration of products was determined by serial dilution method in Columbia broth, followed by heating at 60° C. and plating on Columbia agar to determine the number of preserved spores. Data are presented in table 11.

TABLE 11
Activity of M4 group of products was assessed
against Paenibacillus larvae

| | MIC | Number of viable |
|---|---|---|
| Product | (mcg/ml) | spores in 1 ml |
| M4 | 30.0 | 5.0 |
| M411 | 30.0 | 3.0 |
| M421 | 25.0 | 0.0 |
| M422-1 | 25.0 | 0.0 |
| M431 | 25.0 | 0.0 |
| M441 | 25.0 | 0.0 |
| M451 | 25.0 | 0.0 |
| M461 | 20.0 | 0.0 |
| M481 | 25.0 | 0.0 |

Surprisingly, the data obtained indicate that all products of the M4 group, (although the concentration of M4 component was the same as in “M4”), possess superior activity compared with M4 product both in terms of MIC and in the number of preserved viable spores.

### Example 14. Treatment of Bees Infected with P. larvae

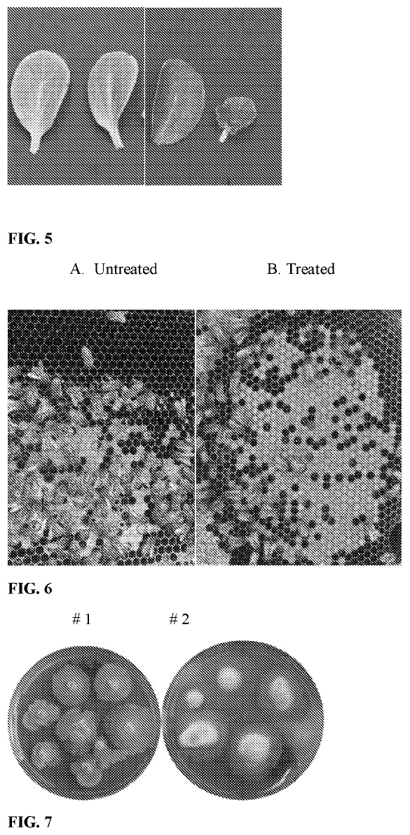

For the tests, an apiary was used for 4 families, of which 2 experimental groups were formed. The treatment was carried out with the product M421, 0.5%. M421 was introduced into the nests of experimental colonies of bees in an apiary by feeding in the composition of sugar syrup (1:1). The concentration of the product was 280 μg/ml. The syrup was given to bees twice with an interval of 2 days at the rate of 100-120 ml per frame. The second group of bee colonies served as a control. Examination of the apiaries revealed a clinically pronounced manifestation of P. larvae infection—(FIG. 5a). In the group treated with M421, the number of honeycombs with infected bees was significantly less, and the number of honeycombs with honey was much higher (FIG. 5b, Table 12).

TABLE 12
Dynamics of changes in the number of affected larvae

| | |
|---|---|
| Number of days from the | Number of affected larvae, pcs. |

| beginning of the experiment | Control | M421 |
|---|---|---|
| 0 | 51 | 54 |
| 10 | 59 | 21 |
| 20 | 67 | 11 |
| 30 | 71 | 2 |

The results obtained indicate 96.2% effectiveness of the use of M4 group of products for the treatment of bee colonies infected with P. larvae (by the number of affected larvae).

### Example 15. Effect of M4 Group of Products on Fish Pathogens

### Fungi of the Saprolegnia spp.

Fungi in the amount of 10e8 cells were added to 1.0 ml of a solution of the test substance and after incubation were washed by PBS with centrifugation, resuspended in buffer, and plated on potato agar to assess survival.

TABLE 13
The effectiveness of the products on
fungi of the genus Saprolegnia

| | Time (minutes) to reach 100% inactivation | | | | |
|---|---|---|---|---|---|
| Product | 0.5% | 0.1% | 0.05% | 0.01% | 0.005% |
| M4 | 1 | 5 | 15 | 15 | 60 |
| ZnSO4 0.01% | —* | — | — | — | — |
| M421 | 1 | 2.5 | 12 | 14 | 48 |
| ZnSO4 0.01% + | — | — | — | — | — |
| sorbitol 0.01 | | | | | |
| μg/mL | | | | | |
| M434-2 | 1 | 3 | 10 | 12 | 45 |
| Na₂HPO4 | — | — | — | — | — |
| (0.01%) | | | | | |
| M461 | 1 | 3 | 10 | 10 | 47 |
| Mannit | — | — | — | — | — |
| (0.01%) | | | | | |
| M481 | 1 | 3 | 13 | 12 | 52 |

*No antimicrobial activity

It can be seed that the tested products M421, M432-2, M 461, M481 possess more pronounced antifungal activity compared with M4. Since the concentration of M4 in these products was the same as in “M4” and excipients do not possess antimicrobial activity, these results point out on bioactive effects of tested compounds

### Example 16. Effect of M4 Group of Products on Fish Eggs

Fish (Trout) fish eggs contaminated with various pathogens was used.

Culture media used:
No. 1 Fish Peptone Agar http://himedialabs.com/TD/RM2580.pdf)+nystatin bacteria control
No. 2 Potato-dextrose agar+streptomycin+gentamicin+penicillin

Results are shown in FIG. 6 and table 13.

Thus, the fish eggs grown on media for bacteria and fungi that infected with a mixture of bacteria and fungi. The fungi are identified as Saprolegnia.

TABLE 14
Effects of tested products on fish eggs

| Product/minimum | | | |
|---|---|---|---|
| concentration causing | | | Growth |
| microbicidal action | Incubation | | after plating |
| M4 50 μg/ml | 15 | minutes | No growth |
| M421 10 μg/ml | 15 | minutes | No growth |
| M431 10 μg/ml | 15 | minutes | No growth |
| M451 10 μg/ml | 15 | minutes | No growth |
| M461 10 μg/ml | 15 | minutes | No growth |
| Methylene blue 1000 mcg/ml | 6 | hours | Lawn |
| Malachite green 100 μg/ml | 30 | minutes | Lawn |

The products M421, M431, M451 and M461 have the greatest activity in disinfecting the game. Surprisingly, that although excipients used in M4 group of products did not have, antimicrobial activity, the effects of M421, M421, M451, M461 were higher than that of M4.

### Example 17. Comparison of the Effectiveness of Standard Products and Product M421 in Fish Eggs Processing

Methylene blue mode—6 hours and Malachite green for 60 minutes and M421 for 15 minutes.

The effect is confirmed by the data shown in FIG. 7

The data obtained indicate that only the treatment with the claimed products makes it possible to achieve rapid disinfection of fish eggs

### Example 18. Effects of Tested Compounds on Fish Treatment

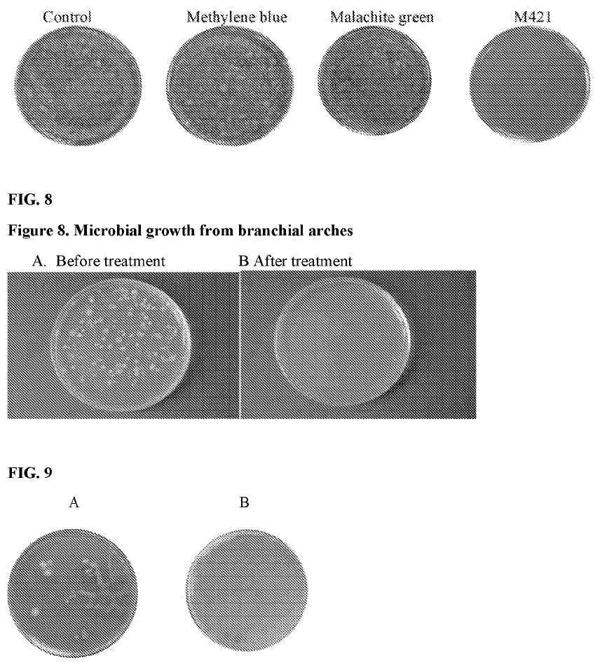

Rainbow trout were treated with M461 0.1%—30 minutes, outside running water. Washings from the branchial arches was done onto potato-dextrose agar http://himedialabs.com/TD/RM2580.pdf) (FIG. 8).

The treatment carried out allowed to completely remove the infection from the gill arches of fish.

### Example 19. Water Treatment

Water from fish breeding pools with a capacity of 5000 liters with a high fish population density. Samples were taken with a sterile sampler. Samples (1.0 ml) were processed by adding M431 at a final concentration of 0.025% and incubated at room temperature for 15 minutes. Then, 10.0 ml of water was added to the sample to dilute the product, and then 100 μl per 1 plate was applied to the agar surface, while the volume of the medium in the plate was 20 ml. Data are presented in FIG. 9.

The results point out that no microbial growth was observed after the treatment with M431

### Example 20. Effect of Compounds on Feed Processing

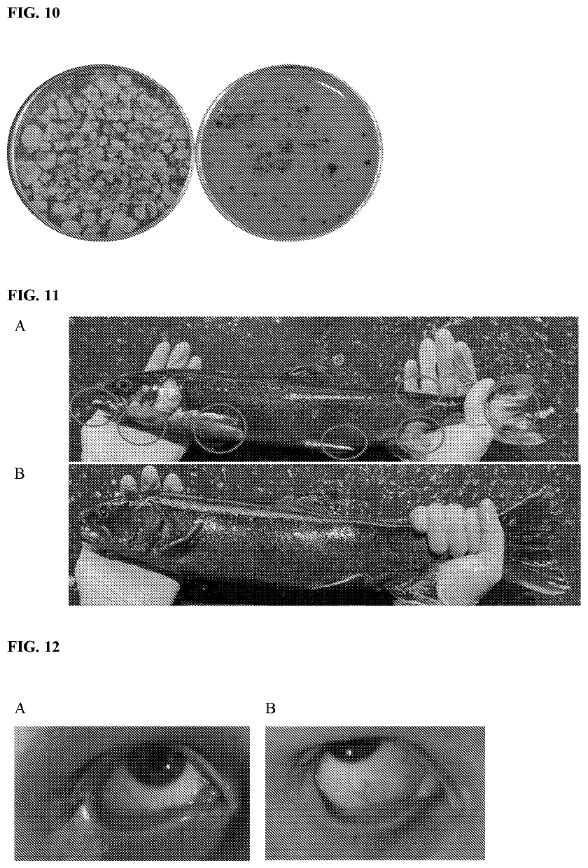

Animal feed in the amount of 2.0 g was treated with M421 0.5%, applied by aerosol method (5 doses of 1000 μg of the product each). Thirty minutes after treatment, the feed was sown on a nutrient medium in Petri dishes. Data are presented in FIG. 10.

As a result, the initially infected feed lost all microbial contamination. Feed processing allows to get rid of dangerous contamination and reduce the risk of contamination of animals and water bodies.

### Example 21. Effect of Tested Compounds on Fish Treatment

Fish—Arctic char from the breeding pool infected with fungi of the genus Saprolegnia were weekly treated with M421 (FIG. 11). before and after three weeks of weekly treatment with (M4 0.1%).

Areas affected by fungi are marked with red.

Processing allows to achieve complete cleansing of the fish body from fungi.

### Example 22. The Use of Compounds of M4 Group for the Treatment of the Acute Conjunctivitis

Totally 18 patients with conjunctivitis were enrolled, complaining on paIn the eyes, itching, discharge from the conjunctival cavity and photophobia. Microbiological examination showed the presence of bacteria in the discharge from the conjunctival cavity, including Actinomyces oris, Streptococcus gordonii, Pseudomonas oryzihabitans Cemella haemolysans, Streptococus spp, Staphylococcus spp and other.

In the discharge from the conjunctival cavity in 4 out of 8 patients, DNA of adenoviruses was detected by real-time polymerase chain reaction.

Patients were treated with M4 (0.01%) or M491 (contained M4 0.01%), that were administered 2 times a day for 1 or 3 days. Therapeutic effect was assessed 24 hours after the last administration of the drug. Data are presented in table 15, and FIG. 12.

TABLE 15
The use of compounds of M4 group for the
treatment of the acute conjunctivitis

| | Nof patients in the group/N of patients |
|---|---|
| Group | with no symptoms of the disease |
| M4 2 times a day for 1 day | 4/1 |
| Sodium hyaluronate (0.1%)* | 2/0 |
| M4 times a day for 3 days | 4/4 |
| M491 2 times a day for 1 day | 4/4 |
| M491 2 times a day for 3 | 4/4 |
| days | |

*patients of this group were later switched for the treatment of M491

The result of using the drug: elimination of symptoms (pain in the eyes, itching, discharge from the conjunctival cells, including adenovirus, prevention of complications and the spread of the process to other parts of the eye.

As can be seen from the data presented, surprisingly M491 had a more pronounced and rapid onset of the therapeutic effect. This fact is surprising and unexpected, since the content of M4 in M491 is the same as in M4, and the excipients have not produced any therapeutic effect. These results point out the 10e5 bioactive effects of the M4 group of products.

### Example 23. The Use of Compounds of M4 Group for the Treatment of Seborrheic Dermatitis

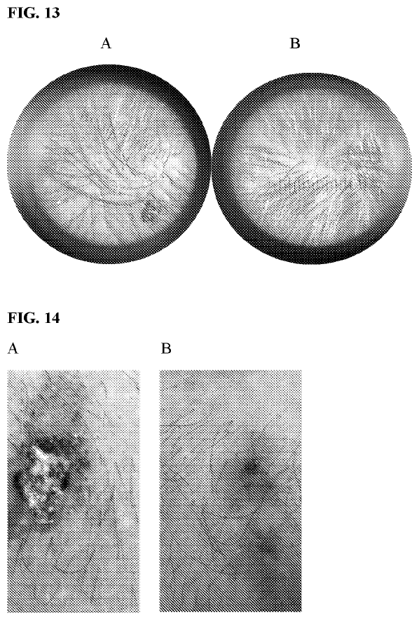

Totally 20 patients with seborrheic dermatitis were enrolled, suffering from this condition for at least 12 months and not using any drugs to treat this condition for the last 14 days. Patients applied M4 or M421 one or two times a day on the affected parts of the head. The efficacy of the drugs was assessed within 14 days, using objective and subjective measures (the prevalence of the disease, the degree of inflammation and infiltration of skin elements, the severity of itching, peeling and crusting). Dermatoscopy was done using the Heine, mini 3000 LED. Data are presented in table 16 and FIG. 13. These results point out the 10e5 bioactive effects of the M4 group of products.

TABLE 16
Effects of tested compounds on seborrheic dermatitis

| | Day at which prevalence of the |
|---|---|
| Group | symptoms was reduced by 80% |
| M4 1 time a day | 12 |
| M4 2 times a day | 9 |
| ZnSO4 0.01% | >14 |
| M421 1 time a day | 7 |
| M421 2 times a day | 7 |

After the treatment, regression of rashes, elimination of itching, and resumption of hair growth in the foci were registered. As can be seen from the data presented, surprisingly M421 had a more pronounced and rapid onset of the therapeutic effect. This fact is surprising and unexpected, since the content of M4 in M421 is the same as in M4, and the excipients have not produced any therapeutic effect. These results point out the bioactive effects of the M4 group of products.

### Example 24. The Use of Compounds of M4 Group for the Treatment of Diabetic Ulcers

Total six patients were enrolled in the study with the diagnosis diabetic foot ulcer/diabetic leg ulcer (DFU). These patients had unhealed ulcers for over 6 months, with the previous unsuccessful experience in using antimicrobial agents, but not using any drugs for the treatment of DFU for the last 14 days. Patients applied M4 and M421 on the ulcer surface topically, two times a day. The efficacy of the drugs was assessed on day 30 based on the dynamics of the clinical symptoms and subjective symptoms (size of the ulcer, intensity of the pain syndrome). Data are presented in table 17 and FIG. 14.

TABLE 17
The use of the tested compounds for
the treatment of diabetic ulcers

| | Percent area reduction of | Day when the pain |
|---|---|---|
| Group | the ulcer on day 30 (%) | syndrome disappeared |
| M4 | 75.33 ± 4.9 | 8.33 ± 0.47 |
| M421 | 100 | 4.66 ± 1.24 |

Cleansing and healing of ulcers, epithelialization of the zone of ulcerative lesions were registered.

As can be seen from the data presented, surprisingly M421 had a more pronounced and rapid onset of the therapeutic effect. This fact is surprising and unexpected, since the content of M4 in M421 is the same as in M4, and the excipients have not produced any therapeutic effect. These results point out the 10e5 bioactive effects of the M4 group of products.

### Example 25. The Use of Compounds of M4 Group for the Treatment of Psoriasis

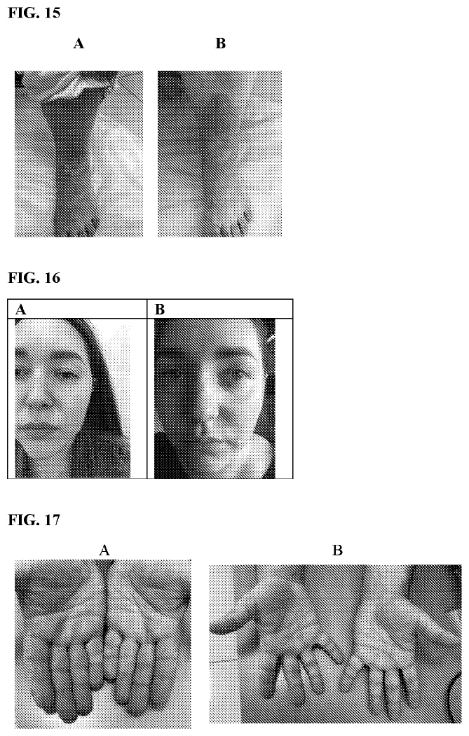

Nine patients with exacerbation of psoriasis, suffering from this disease for at least over 7 years were enrolled. All patients have not been using any anti-psoriatic drugs for the last 14 days. M4, M421 or M491 were applied two times a day on affected areas. The efficacy of the drugs was assessed by the dynamics of PASI score (intensity of Erythema, Induration, Desquamation). Data are presented in table 18 and FIG. 15.

TABLE 18
The use of the tested compounds for the psoriasis treatment

| Group | Day 0 | Day 7 | % decrease |
|---|---|---|---|
| M4 | 8.2 ± 1.2 | 3.3 ± 1.24 | 60 |
| M421 | | 0.81 ± 0.66 | 98 |
| M491 | | 2.3 ± 0.47 | 72 |

A decrease of clinical manifestations of the disease, the elimination of pathological subjective sensations, an improvement in the patient's quality of life were registered.

As can be seen from the data presented, surprisingly M421 had a more pronounced and rapid onset of the therapeutic effect. This fact is surprising and unexpected, since the content of M4 in M421 is the same as in M4, and the excipients have not produced any therapeutic effect. These results point out the 10e5 biostimulating effects of the M4 group of products.

Psoriasis is not a microbial disease and has been linked to deficiencies in the immune system. In this regard, the obtained clinical effect is associated precisely with the bioactive activity of the claimed drugs.

### Example 26. The Use of Compounds of M4 Group for the Treatment of Contact Dermatitis

Patient, woman 34 years old. Suffers for 3 days for contact dermatitis without treatment. Complaints: redness, oozing, manifestations of the disease; in the area of the cheek nose, rashes in the form of large-lamellar peeling, small vesicles, yellowish discharge hyperemia of the skin.

The treatment was done with M421 in the soap 2 times a day. Lotions with M421 were applied to the affected area 2 times a day for 7 days. Data are presented in FIG. 16.

It is seen that the use of the compound led to the cessation of disease progression, reduction of itching, and resolution of rashes were registered. A similar effect confirms the bioactive effect of the drug.

### Example 27. The Use of Compounds of M4 for the Treatment of Eczema

Six patients were enrolled in the study. All patients had an eczema, suffering form this disease over 6 months and not using any medications for its treatment of the last 14 days. Patients applied M4 and M421 two times a day on the affected surface areas. The efficacy of the drugs was achieved within 30 days, based on the dynamics of the clinical symptoms and subjective symptoms (spread of the lesions, epithelization). Data are presented in table 19 and FIG. 17.

TABLE 19
The use of the tested compounds for the treatment of eczema

| | Day therapeutic effect |
|---|---|
| Group | is achieved |
| M4 | 28 |
| M421 | 17 |

Patient: man, 55 years old. Diagnosis—Dyshidrotic eczema, exacerbation. The duration of the disease—10 years. The main complaints are itching, burning, pain the area of cracks. Objectively, there are multiple large-lamellar peeling in the area of the hands, cracks up to 1-2 cm in length, not epithelialized, single vesicles. The drug M421 0.5% was used in the form of a solution: 2-3 times a day in the form of rubbing with a cotton swab, once a day in the form of a gel containing additionally lightly crosslinked acrylic polymers, in the area of the brushes, in particular, in the area of cracks.

As a result of the use of the tested drugs, the condition improved significantly. The progression of the disease was stopped, the itching decreased, the rashes were resolved, the cracks closed. In addition, no exacerbations were recorded after treatment for three months (observation time).

As can be seen from the data presented, surprisingly M421 had a more pronounced and rapid onset of the therapeutic effect. This fact is surprising and unexpected, since the content of M4 in M421 is the same as in M4, and the excipients have not produced any therapeutic effect. These results point out the 10e5 bioactive effects of the M4 group of products.

### Example 28. The Use of Compounds of M4 for the Treatment of Herpes Zoster

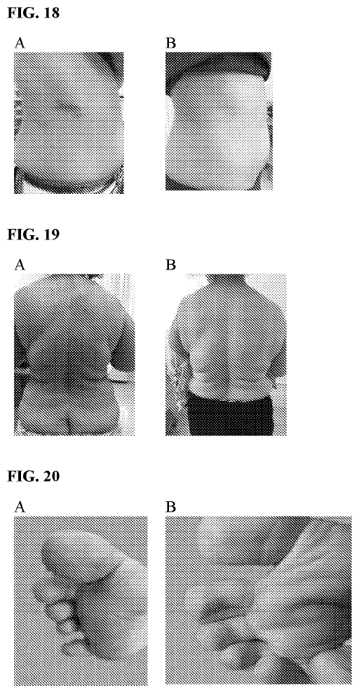

Six patients with herpes zoster were included in the study, with the diseases manifested within the last 3 day, prior to any treatment. M4 and M491 were applied topically three times a day on the affected parts. The efficacy of the drugs was assessed within 7 days by the dynamics of, clinical symptoms disappearance (day of rush disappearance, itching). Data are presented in table 20 and FIG. 18.

TABLE 20
The use of the tested compounds for the treatment of Herpes zoster

| | Day of rush |
|---|---|
| Group | disappearance |
| M4 | 8 |
| M491 | 4 |

Data received that the tested compounds resulted in the rapid and significant improvement of patient's condition decrease of the itching, burning sensation, the appearance of new rashes was arrested and the remaining crus resolved quickly

As can be seen from the data presented, surprisingly M491 had a more pronounced and rapid onset of the therapeutic effect. This fact is surprising and unexpected, since the content of M4 in M491 is the same as in M4, and the excipients have not produced any therapeutic effect. These results point out the bioactive effects of the M4 group of products.

### Example 29. The Use of Compounds of M4 for the Treatment of Pompholix (Dyshidrosis)

Patient, woman 35 years old. Diagnosis Hyperhidrosis of the hands and feet. Suffering for more than 10 years, currently using aluminum-containing products (DryDry). Complaints: unpleasant odor from the body and the cloths, intense sweating after stressful situations.

Treatment. The first 5 days M491 solution, treatment of hands and feet 3 times a day, treatment of M421 shoes in the morning before use and in the evening after. Then 5 days: solution, 2 times a day the same. Then 4 days: then treatment of hands and feet once a day, shoe treatment every other day.

The result of treatment is the termination of the progression of the disease, reduction of itching, restoration of normal skin moisture.

### Example 30. The Use of Compounds of M4 for the Treatment of Folliculitis of the Chest and Back

Six patients were enrolled in the study with the folliculitis of the chest and back. Patients were treated with the M4 and M421 two times a day, that were applied topically on the surface of the affected regions. The efficacy of the drugs was assessed in 7 days, based on the dynamics of the clinical symptoms and subjective symptoms (area, itching). Data are presented in table 21 and FIG. 19.

TABLE 21
The use of the tested compounds for the treatment of Folliculitis

| | Day of clinical symptoms |
|---|---|
| Group | disappearance |
| M4 | 7 |
| M421 | 4 |

Treatment drug M491 solution: rubbed with a cotton swab 2-3 times a day. Duration of treatment 7 days

Patient, a 57-year-old woman. She suffers for 5 days, she has not been treated on her own. Complaints: spreading rashes in the chest, back rashes in the form of multiple pustular rashes, rounded, erythematous spots.

As can be seen from the data presented, surprisingly M421 had a more pronounced and rapid onset of the therapeutic effect. This fact is surprising and unexpected, since the content of M4 in M491 is the same as in M4, and the excipients have not produced any therapeutic effect. These results point out the bioactive effects of the M4 group of products.

### Example 31. The Use of Compounds of M4 for the Treatment of Epidermophytosis

Patient, woman, 22 years old. Diagnosis of Epidermophytosis of the feet. She has been ill for two weeks. Manifestation of the disease: multiple papulo-vesicular rashes in the toes on the inner side of the foot. In the area of both feet there are papular, vesicular rashes, multiple, small-lamellar peeling.

Treatment: lotions, M491, 0.5% 2 times a day for 30 minutes, within 7 days (FIG. 20).

The results clearly show high efficacy of M4 group of products for the treatment of epidermophytosis.

### Example 32. The Use of Compounds of M4 Group for the Treatment of Sinusitis

Six patients with acute sinusitis were enrolled in the study. They were treated with a one-time instillation in the sinus of M4 or M431. The efficacy of the drugs was assessed on day 30 based on the dynamics of the clinical symptoms. Data are presented in table 22.

TABLE 22
The use of compounds of M4 for the treatment of sinusitis

| | Day of clinical symptoms |
|---|---|
| Group | disappearance |
| M4 | 1.66 ± 0.47 |
| M431 | 1 ± 0 |

Data received indicate that M4 and M431 resulted in quick therapeutic effect. As it can be seen As can be seen from the data presented, surprisingly M31 had a more pronounced and rapid onset of the therapeutic effect. This fact is surprising and unexpected, since the content of M4 in M491 is the same as in M4, and the excipients have not produced any therapeutic effect. These results point out the bioactive effects of the M4 group of products.

### Example 33. Inactivation of M4 Group of Products

Alginate, Carboxymethylcellulose, Staphylococcus aureus VT209 test were used as inactivators for M421.

The inactivator was added to the drug solution and after centrifugation of 4,000 g 15 min, the antimicrobial activity of the supernatant was determined. Data are presented in table 23.

TABLE 23
The use of compounds of M4 group of products inactivation

| | Minimal inhibitory concentration (mcg/ml) | | |
|---|---|---|---|
| M421/inactivator | | | M421/carboxymethyl |
| ratio | M421 | M421/alginate | cellulose |
| 1:0 | 0.5 | — | — |
| 1:1 | — | 0.5 | 4.0 |
| 1:2 | — | 0.5 | 31.0 |
| 1:10 | — | 0.5 | 125.0 |
| 1:50 | — | 16 | 125.0 |

The highest neutralization activity is shown for carboxymethylcyllulose

### Example 34. The Use of Compounds of M4 Group for the Treatment of Complicate Caries

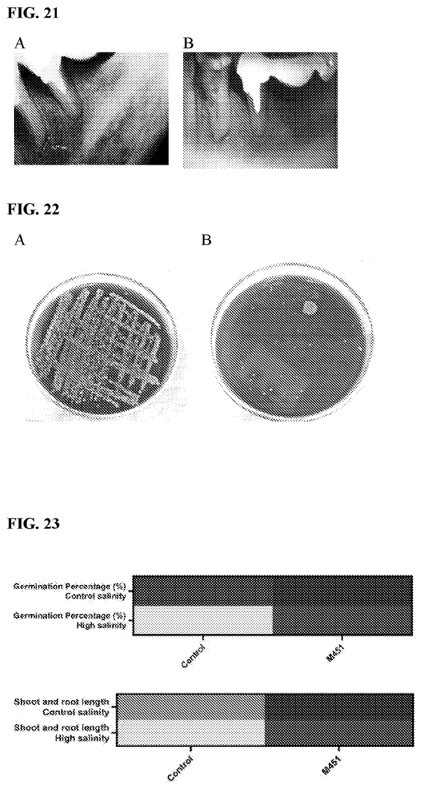

Six patients with complicated caries and cyst were enrolled in the study. Patients were treated with M4 or M421 which were used to wash the cyst through the canal and gel with 0.3% of M4 or, M421 as a temporary filling was installed. This procedure was repeated once in every 10 days. The efficacy of the drugs was assessed on day 30 based on the dynamics of the clinical symptoms, disappearance of the inflammatory focus and replacement with bone tissue. Data are presented in table 24 and FIG. 21

TABLE 24
The use of compounds of M4 for the treatment of complicate caries

| | Treatment cycle at which the symptoms |
|---|---|
| Group | of the disease disappear |
| M4 | 5.33 ± 0.47 |
| M421 | 4.33 ± 0.47 |

The use of M4 group of products were highly effective for the treatment of complicated caries. As can be seen from the data presented, surprisingly M421 had a more pronounced and rapid onset of the therapeutic effect. This fact is surprising and unexpected, since the content of M4 in M491 is the same as in M4, and the excipients have not produced any therapeutic effect. These results point out the bioactive effects of the M4 group of products.

### Example 35. The Use of Compounds of M4 Group for the Treatment of Periodontitis

Six patients were enrolled in the study with the diagnosis periodontitis. Patients were treated with M4 and M421 in the form of gel with containing lightly crosslinked acrylic polymers. Compounds were administered inside the periodontal pocket during three visits to a doctor and daily after the teeth brushing. Data are presented in table 25.

TABLE 25
The use of compounds of M4 group for
the treatment of periodontitis

| | Depth of periodontal | Depth of periodontal |
|---|---|---|
| | pocket before the | pocket after the |
| Group | treatment | treatment |
| M4 | 5.5 + 0.5 | 2.7 + 0.47 |
| M421 | | 1.33 + 0.47 |

As can be seen from the data presented, both M421 and M4 possess potent anti-periodontitis activity. Surprisingly M491 had a more pronounced and rapid onset of the therapeutic effect. This fact is surprising and unexpected, since the content of M4 in M491 is the same as in M4, and the excipients have not produced any therapeutic effect. These results point out the bioactive effects of the M4 group of products.

### Example 36. The Use of Compounds of M4 Group for the Treatment of Cystitis

Six patients with acute cystitis were enrolled in the study. M4 and M21 were used for bladder instillations. The efficacy was assessed based on the number of instillations required to reach therapeutic effect. Data are presented in table 26.

TABLE 26
The use of compounds of M4 group for the treatment of cystitis

| | Number of bladder instillations required |
|---|---|
| Group | to reach therapeutic efficacy |
| M4 | 2.33 + 0.47 |
| M421 | 1.33 + 0.47 |

Both M4 and M421 worked for the treatment of cystitis.

As can be seen from the data presented, surprisingly M421 had a more pronounced and rapid onset of the therapeutic effect. This fact is surprising and unexpected, since the content of M4 in M491 is the same as in M4, and the excipients have not produced any therapeutic effect. These results point out the bioactive effects of the M4 group of products.

### Example 37. The Use of Compounds of M4 Group for the Treatment of Hoof Rot

The preparation of M421 in the form of a gel was applied to the affected areas for 5 days

The result of treatment is the complete disappearance of the signs of the disease and its manifestations in the behavior of the animal. The hooves looks supple and healthy. Microbiological study showed a dramatic reduction in microbial infection (FIG. 22)

These results clearly show that M4 group of products are highly effective for the treatment of hooves infection.

### Example 38. Effect of Complex M451 on Seeds Growth in Soil Salinity

We studied how plating seeds pretreated with M451 affected plant growth at higher soil salinity. For that seeds of Triticale (x Triticosecale Wittmack) were spread and allowed to grow on Potato dextrose agar with 0 (deionized water, as a control) and 250 mM salt (MgSO4) in a 9-cm-diam Petri dish. Seeds were pretreated with M451 from 10 to 5000 μg/ml. M451 was washed out and cells were placed in growth chamber at 25±1° C. with 12 h daylight. Daily observation and counting of the number of seeds which were sprouted and germinated were done up to 7 days. Sprouted seeds were referred to the seeds which have reached the ability to produce at least one noticeable plumule or radicle. Seeds were considered germinated with at least 2 mm radicle emergence from the seed coat. After seven days of treatment application, measurement of parameters was done and calculated.

Seeds were transplanted into plastic nursery pot for plants (L×W×D of 3.25″×2.75″×2.75″) filled with a mixture of soil and peat moss (3:1, v/v) containing organic fertilizer. The temperature of the greenhouse was maintained at 25±2° C. and 10±2° C. during day and night, respectively. Each treatment consisted of three replicates and 1/100 plant were planted per plastic pot. At harvest, after treatment, plant growth parameters, were measured. The represented values were shown as mean±SE with a minimum of three independent replicates (n=3). Data are presented in FIG. 23.

1. The product to be used in agriculture veterinary, medicine including M4 (Poly-N1-hydrazino (imino) methyl-1,6-hexanediamine and/or compositions of the M4 and zinc and/or glycerin and/or sorbitol and/or boron and/or mannitol salts and/or Sodium hydrogenphosphate and/or Sodium dihydrogen phosphate and/or manganese salts and/or glutamic acid hyaluronic acid and/or inhibitors of reverse transcription and/or integration/recombination and/or proteases, and/or DNAse and/or RNAse, and/or DNAse+RNAse and/or VTL having a bioactive effect and/or the ability to control the activity of cells and/or to act on the properties of cells and/or group of cells controlled by DNA and/or RNA receptors of cells to improve the properties of plants and/or animals and/or the properties of water and/or soil, as well as the prevention and/or treatment of diseases of plants, animals and humans.

2. The method of increasing germination, intensity and growth rate, stress tolerance, product and grain yield, chlorophyll formation and productivity of plants, increasing the productivity of aquacultures, fertilizing fish, cultivating soil, water, breeding aquatic animals and aquariums, increasing the safety of feed for farm animals and aquacultures, preventing and treating diseases and condition management (or condition correction) of plants, animals and people.

3. The product according to claim 1 are fry forms, powders, solutions, gels and/or emulsions and/or ointments, additionally including hydrophilic ointment bases, including lightly crosslinked acrylic polymers, and/or lipophilic hydrocarbon, fatty, silicone and other components.

4. The product according to claim 1, wherein the product is a complex of DNAse (from 0.1 μg/ml to 500.0 μg/ml), and/or RNAse (from 0.1 μg/ml to 500.0 μg/ml), and/or DNAse+RNAse (from 0.1 μg/ml to 500.0 μg/ml) and/or reverse transcription inhibitors (from 0.1 μg/ml to 5000.0 μg/ml), and/or integration/recombination inhibitors (from 0.1 μg/ml to 5000.0 μg/ml), and/or proteases (from 0.1 μg/ml to 5000.0 μg/ml), intended for the prevention and treatment of animals and humans.

5. The product according to claim 1, wherein the product is M4 in an amount from 0.001 μg/ml to 10e5 μg/ml or compositions of M4 and zinc salts (from 0.1 μg/ml to 5000.0 μg/ml) of glycerol (from 0.1 μg/ml to 5000.0 μg/ml) sorbitol (from 0.1 μg/ml to 5000.0 μg/ml), boron salts (from 0.1 μg/ml to 5000.0 μg/ml), glutamate salts (from 0.1 μg/ml to 5000.0 μg/ml), mannitol (from 0.1 μg/ml to 5000.0 μg/ml), salts of hydrogen phosphates (from 0.1 μg/ml to 5000.0 μg/ml), salts of dihydrophosphates (from 0.1 μg/ml to 5000.0 μg/ml) salts of manganese (from 0.1 μg/ml to 5000.0 μg/ml), glutamic acid salts (from 0.1 μg/ml to 5000.0 μg/ml), or reverse transcription inhibitors (from 0.1 μg/ml to 5000.0 μg/ml), integration/recombination (from 0.1 μg/ml to 5000.0 μg/ml), proteases (from 0.1 μg/ml to 5000.0 μg/ml), DNAse (from 0.1 μg/ml to 500.0 μg/ml), RNAse (from 0.1 μg/ml to 500.0 μg/ml), DNAse+RNAse (from 0.1 μg/ml to 500.0 μg/ml) and complexes of the above products, which are used to increase the productivity of agricultural and/and whether ornamental and/or forest and/or domestic plants and/or aquaculture are used for the regulation of the plants properties with a non-limiting examples to accelerate/enhance growth rate, propagation, breeding, productivity, germination rate, flowering, modulating flowering in plants (acceleration and/or delaying the time to flowering) and/or increasing and/or decreasing the duration of flowering, increasing of organ size, vigor, photosynthetic area, number of leaves, flowers and/or plants root length, number of pods, improve the tillering, flowability and plantability, blooming, safety of crops in plants, plant height, cations content increases, biomass increases, sprout growth increases, increased grain yield, more early and sprouting and fruiting, increased/or altered Oil, starch, protein, Nutrients, Vitamins, fatty-acids, amino acids, sugars, plant weight, fiber length modulate senescence, increasing number of plants capable of growing in a given area ameliorates negative effects of hypoxia, darkness, drying, flooding, cold, soil salinity, nutrient or mineral or nitrogen deficiency, stress tolerance to other negative biological, chemical and physical effects

6. The product according to claim 1, wherein the product M4 in an amount from 0.001 μg/ml to 10e5 μg/ml and/or a composition of M4 and zinc and/or glycerol and/or sorbitol and/or mannitol and/or sodium hydrogen phosphate and/or sodium dehydrogen phosphate and/or salts of boron and/or glutamate and/or manganese, which is used for the treatment of soil and/or water bodies to increase the productivity of agricultural and/or ornamental and/or forest and/or domestic plants and/or aquaculture

7. The product according to claim 1, wherein the product is a complex of M4 in an amount from 0.001 μg/ml to 10e5 μg/ml and zinc salt (from 0.1 μg/ml to 5000.0 μg/ml), and/or glycerol (from 0.1 μg/ml to 5000.0 μg/ml) sorbitol (from 0.1 μg/ml to 5000.0 μg/ml), boron salts (from 0.1 μg/ml to 5000.0 μg/ml), glutamate salts and/or (from 0.1 μg/ml to 5000.0 μg/ml), and/or mannitol (from 0.1 μg/ml to 5000.0 μg/ml), and/or salts of hydrogen phosphates (from 0.1 μg/ml to 5000.0 μg/ml), and/or salts of dihydrogen phosphates (from 0.1 μg/ml to 5000.0 μg/ml) and/or manganese salts (from 0.1 μg/ml to 5000.0 μg/ml), and/or glucuronic acid soda (from 0.1 μg/ml to 5000.0 μg/ml) which is used to increase the productivity of agricultural and/or forest and/or domestic plants and/or ornamental and/or aquaculture due to seed dressing and/or treatment of roots and/or vegetative parts of the plant.

8. The product according to claim 1 that is used for the regulation of plants' growth within farms (including vertical farms) of selected from the group consisting of a tree, a herb, a bush, a grass, a vine, a fem, moss and, a green algae, a monocotyledonous plant, and a dicotyledonous plant, including wheat, soy, rice, sugar cane, potato, barley, maize, oat, rice, sorghum, sugar cane, tomato, hybrid plants, new plants sugarcane, corn, cotton, grapes, bananas, cassava, beans, nuts, oil crops etc.

9. The product according to claim 1, wherein the product is a complex of M4 in an amount from 0.001 μg/ml to 10e5 μg/ml, zinc salts (from 0.1 μg/ml to 5000.0 μg/ml) and DNAse (from 0.1 μg/ml to 500.0 μg/ml), which is used to increase the productivity of agricultural and/or ornamental and/or forest and/or domestic plants and/or aquacultures by treating seeds and/or treating the roots and/or vegetative parts of the plant.

10. The product according to claim 1, wherein the product is a complex of M4 in an amount from 0.001 μg/ml to 10e5 μg/ml, zinc salts (from 0.1 μg/ml to 5000.0 μg/ml) and RNAse (from 0.1 μg/ml to 500.0 μg/ml), which is used to increase the productivity of agricultural and/or ornamental and/or forest and/or domestic plants and/or aquacultures by treating seeds and/or treating the roots and/or vegetative parts of the plant.

11. The product according to claim 1, wherein the product is a complex of M4 in an amount from 0.001 μg/ml to 10e5 μg/ml, zinc salts (from 0.1 μg/ml to 5000.0 μg/ml), DNAse (from 0.1 μg/ml to 500.0 μg/ml) and RNAse (from 0.1 μg/ml to 500.0 μg/ml), which is used to increase the productivity of agricultural and/or ornamental and/or forest and/or domestic plants and/or aquaculture

12. The product according to claim 1, wherein the product is a complex of M4 in an amount from 0.001 μg/ml to 10e5 μg/ml, zinc salts (from 0.1 μg/ml to 5000.0 μg/ml) and DNAse (from 0.1 μg/ml to 500.0 μg/ml) and Raltegravir (from 0.1 μg/ml to 5000.0 g/ml), which is used to increase the productivity of agricultural and/or ornamental and/or forest and/or domestic plants and/or aquaculture through seed treatment and/or root treatment and/or vegetative parts of the plant.

13. The product according to claim 1, wherein the product is a complex of DNAse (from 0.1 μg/ml to 500.0 μg/ml) or RNAse (from 0.1 μg/ml to 500.0 μg/ml) or DNAse mRNAse (from 0.1 μg/ml to 500.0 μg/ml) and Raltegravir (from 0.1 μg/ml to 5000.0 μg/ml), which is used to increase the productivity of agricultural and/or ornamental and/or forest and/or domestic plants and/or aquaculture through seed treatment and/or root treatment and/or vegetative parts of the plant.

14. The product according to claim 1, wherein the product is a complex of DNAse (from 0.1 μg/ml to 500.0 μg/ml) or RNAse (from 0.1 μg/ml to 500.0 μg/ml) or DNAse+RNAse (from 0.1 μg/ml to 500.0 μg/ml) and protease inhibitors (lopinavir/ritonavir) (from 0.1 μg/ml to 5000.0 μg/ml), which is used to increase the productivity of agricultural and/or ornamental and/or forest and/or domestic plants and/or aquaculture through seed treatment and/or root treatment and/or vegetative parts of the plant.

15. The product according to claim 1, wherein the product is a complex of M4 (0.001 μg/ml to 10e5 μg/ml and/or DNAse (from 0.1 μg/ml to 500.0 μg/ml) or RNAse (from 0.1 μg/ml to 500.0 μg/ml) or DNAse+RNAse (from 0.1 μg/ml to 500.0 μg/ml) and protease inhibitors (lopinavir/ritonavir) (from 0.1 μg/ml to 5000.0 μg/ml), which is used to increase the productivity of agricultural and/or ornamental and/or forest and/or domestic plants and/or aquaculture through seed treatment and/or root treatment and/or vegetative parts of the plant.

16. The product according to claim 1, wherein the product is M4 in an amount from 0.001 μg/ml to 10e5 μg/ml or compositions of M4 and zinc salts (from 0.1 μg/ml to 5000.0 μg/ml) of glycerol (from 0.1 μg/ml to 5000.0 μg/ml) sorbitol (from 0.1 μg/ml to 5000.0 μg/ml), boron salts (from 0.1 μg/ml to 5000.0 μg/ml), glutamate salts (from 0.1 μg/ml to 5000.0 μg/ml), mannitol (from 0.1 μg/ml to 5000.0 μg/ml), salts of hydrogen phosphates (from 0.1 μg/ml to 5000.0 μg/ml), salts of dihydrophosphates (from 0.1 μg/ml to 5000.0 μg/ml) salts of manganese (from 0.1 μg/ml to 5000.0 μg/ml), glutamic acid salts (from 0.1 μg/ml to 5000.0 μg/ml), or reverse transcription inhibitors (from 0.1 μg/ml to 5000.0 μg/ml), integration/recombination (from 0.1 μg/ml to 5000.0 μg/ml), proteases (from 0.1 μg/ml to 5000.0 μg/ml), DNAse (from 0.1 μg/ml to 500.0 μg/ml), RNAse (from 0.1 μg/ml to 500.0 μg/ml), DNAse+RNAse (from 0.1 μg/ml to 500.0 μg/ml) and complexes of the listed products that are used for processing fish eggs in order to increase the efficiency of supplementation, sex management and infection control.

17. The product according to claim 1, wherein the product is M4 in an amount from 0.001 μg/ml to 10e5 μg/ml or compositions of M4 and zinc salts (from 0.1 μg/ml to 5000.0 μg/ml), glycerol (from 0.1 μg/ml to 5000.0 μg/ml), sorbitol (from 0.1 μg/ml to 5000.0 μg/ml), boron salts (from 0.1 μg/ml to 5000.0 μg/ml), glutamate salts (from 0.1 μg/ml to 5000.0 μg/ml), mannitol (from 0.1 μg/ml to 5000.0 μg/ml), salts of hydrogen phosphates (from 0.1 μg/ml to 5000.0 μg/ml), salts of dihydrogen phosphates (from 0.1 μg/ml to 5000.0 μg/ml) salts of manganese (from 0.1 μg/ml up to 5000.0 μg/ml), glutamic acid salts (from 0.1 μg/ml to 5000.0 μg/ml), and complexes of the listed products that are used to increase the growth rate of weight gain, and other vital and commercially important characteristics of animals and aquaplankton due to treatment feed.

18. A product according to claim 1, wherein the product is M4 in an amount from 0.001 μg/ml to 10e5 g/ml or compositions of M4 and zinc salts (from 0.1 μg/ml to 5000.0 μg/ml), glycerol (from 0.1 μg/ml to 5000.0 μg/ml), sorbitol (from 0.1 μg/ml to 5000.0 μg/ml), boron salts (from 0.1 μg/ml to 5000.0 μg/ml), glutamate salts (from 0.1 μg/ml to 5000.0 μg/ml), mannitol (from 0.1 μg/ml to 5000.0 μg/ml), salts of hydrogen phosphates (from 0.1 μg/ml to 5000.0 μg/ml), salts of dihydrogen phosphates (from 0.1 μg/ml to 5000.0 μg/ml) salts of manganese (from 0.1 μg/ml up to 5000.0 μg/ml), glutamic acid salts (from 0.1 μg/ml to 5000.0 μg/ml), or reverse transcription inhibitors (from 0.1 μg/ml to 5000.0 μg/ml), integration/recombination (from 0.1 μg/ml to 5000.0 μg/ml), proteases (from 0.1 μg/ml to 5000.0 μg/ml), DNAse (from 0.1 μg/ml to 500.0 μg/ml), RNAse (from 0.1 μg/ml to 500.0 μg/ml), DNAse+RNAse (from 0.1 μg/ml to 500.0 μg/ml) of the complexes of the listed products, as well as their forms, which are gels and/or emulsions and/or ointments and/or sol bores that are used to prevent and treat hoof rot and/or animal skin diseases

19. The product according to claim 1, wherein the product is M4 in an amount from 0.001 μg/ml to 10e5 μg/ml or compositions of M4 and zinc salts (from 0.1 μg/ml to 5000.0 μg/ml), glycerol (from 0.1 μg/ml to 5000.0 μg/ml), sorbitol (from 0.1 μg/ml to 5000.0 μg/ml), boron salts (from 0.1 μg/ml to 5000.0 μg/ml), glutamate salts (from 0.1 μg/ml to 5000.0 μg/ml), mannitol (from 0.1 μg/ml to 5000.0 μg/ml), salts of hydrogen phosphates (from 0.1 μg/ml to 5000.0 μg/ml), salts of dihydrogen phosphates (from 0.1 μg/ml to 5000.0 μg/ml) salts of manganese (from 0.1 μg/ml up to 5000.0 μg/ml), glutamic acid salts (from 0.1 μg/ml to 5000.0 μg/ml), or reverse transcription inhibitors (from 0.1 μg/ml to 5000.0 μg/ml), integration/recombination (from 0.1 μg/ml to 5000.0 μg/ml), proteases (from 0.1 μg/ml to 5000.0 μg/ml), DNAse (from 0.1 μg/ml to 500.0 μg/ml), RNAse (from 0.1 μg/ml to 500.0 μg/ml), DNAse+RNAse (from 0.1 μg/ml to 500.0 μg/ml) of the complexes of the listed products, as well as their forms, which are gels and/or emulsions and/or ointments and/or sol ora, which is used to treat eye diseases with conjunctivitis and dacryocystitis

20. The product according to claim 1, wherein the product is M4 in an amount from 0.001 μg/ml to 10e5 μg/ml or compositions of M4 and zinc salts (from 0.1 μg/ml to 5000.0 μg/ml) of glycerol (from 0.1 μg/ml to 5000.0 μg/ml) sorbitol (from 0.1 μg/ml to 5000.0 μg/ml), boron salts (from 0.1 μg/ml to 5000.0 μg/ml), glutamate salts (from 0.1 μg/ml to 5000.0 μg/ml), mannitol (from 0.1 μg/ml to 5000.0 μg/ml), salts of hydrogen phosphates (from 0.1 μg/ml to 5000.0 μg/ml), salts of dihydrophosphates (from 0.1 μg/ml to 5000.0 μg/ml) salts of manganese (from 0.1 μg/ml to 5000.0 μg/ml), glutamic acid salts (from 0.1 μg/ml to 5000.0 μg/ml), or reverse transcription inhibitors (from 0.1 μg/ml to 5000.0 μg/ml), integration/recombination (from 0.1 μg/ml to 5000.0 μg/ml), proteases (from 0.1 μg/ml to 5000.0 μg/ml), DNAse (from 0.1 μg/ml to 500.0 μg/ml), RNAse (from 0.1 μg/ml to 500.0 g/ml), DNAse+RNAse (from 0.1 μg/ml to 500.0 μg/ml) and complexes of the listed products, which are gels and/or emulsions and/or ointments and/or solutions that are used is used to correct the condition of the oral cavity, including periodontal and endodontic mucosa, as well as cysts and granulomas.

21. The product according to claim 1, wherein the product is M4 in an amount from 0.001 μg/ml to 10e5 μg/ml or compositions of M4 and zinc salts (from 0.1 μg/ml to 5000.0 μg/ml) of glycerol (from 0.1 μg/ml to 5000.0 μg/ml) sorbitol (from 0.1 μg/ml to 5000.0 μg/ml), boron salts (from 0.1 μg/ml to 5000.0 μg/ml), glutamate salts (from 0.1 μg/ml to 5000.0 μg/ml), mannitol (from 0.1 μg/ml to 5000.0 μg/ml), salts of hydrogen phosphates (from 0.1 μg/ml to 5000.0 μg/ml), salts of dihydrophosphates (from 0.1 μg/ml to 5000.0 μg/ml) salts of manganese (from 0.1 μg/ml to 5000.0 μg/ml), glutamic acid salts (from 0.1 μg/ml to 5000.0 μg/ml), or reverse transcription inhibitors (from 0.1 μg/ml to 5000.0 μg/ml), integration/recombination (from 0.1 μg/ml to 5000.0 μg/ml), proteases (from 0.1 μg/ml to 5000.0 μg/ml), DNAse (from 0.1 μg/ml to 500.0 μg/ml), RNAse (from 0.1 μg/ml to 500.0 μg/ml), DNAse+RNAse (from 0.1 μg/ml to 500.0 μg/ml) and complexes of the listed products that are used to correct the condition of the skin, subcutaneous tissue and/and whether the eyes and/or mucous membranes of animals and humans with various diseases, including seborrheic dermatitis, neuroderma, Psoriasis, Herpes, papillomatosis, mycoses, erysipelas, trophic and diabetic ulcers, trauma, acne bedsores, folliculitis, furunculosis, angulitis (seizure) alopecia.

22. The product according to claim 1, wherein the product is M4 in an amount from 0.001 μg/ml to 10e5 μg/ml or compositions of M4 and zinc salts (from 0.1 μg/ml to 5000.0 μg/ml) of glycerol (from 0.1 μg/ml to 5000.0 μg/ml) sorbitol (from 0.1 μg/ml to 5000.0 μg/ml), boron salts (from 0.1 μg/ml to 5000.0 μg/ml), glutamate salts (from 0.1 μg/ml to 5000.0 μg/ml), mannitol (from 0.1 μg/ml to 5000.0 μg/ml), salts of hydrogen phosphates (from 0.1 μg/ml to 5000.0 μg/ml), salts of dihydrophosphates (from 0.1 μg/ml to 5000.0 μg/ml) salts of manganese (from 0.1 μg/ml to 5000.0 μg/ml), glutamic acid salts (from 0.1 μg/ml to 5000.0 μg/ml), or reverse transcription inhibitors (from 0.1 μg/ml to 5000.0 g/ml), integration/recombination (from 0.1 μg/ml to 5000.0 μg/ml), proteases (from 0.1 μg/ml to 5000.0 μg/ml), DNAse (from 0.1 μg/ml to 500.0 μg/ml), RNAse (from 0.1 μg/ml to 500.0 μg/ml), DNAse+RNAse (from 0.1 μg/ml to 500.0 μg/ml) and complexes of the above products that are used to correct the condition of the sinuses and/or urinary mucosa bladder.

23. The method according to claim 2 wherein the soil and/or water bodies in order to increase the productivity of agricultural and/or forest and/or domestic plants and/or aquacultures are treated with the M4 in an amount from 0.001 μg/ml to 10e5 μg/ml or compositions M4 and zinc salts (from 0.1 μg/ml to 5000.0 μg/ml) glycerol (from 0.1 μg/ml to 5000.0 μg/ml) sorbitol (from 0.1 μg/ml to 5000.0 μg/ml), boron salts (from 0.1 μg/ml to 5000.0 μg/ml), glutamate salts (from 0.1 μg/ml to 5000.0 μg/ml), mannitol (from 0.1 μg/ml to 5000.0 μg/ml), hydrogen phosphate salts (from 0.1 μg/ml to 5000.0 μg/ml), dihydrophosphate salts (from 0.1 μg/ml to 5000.0 μg/ml) manganese salts (from 0.1 μg/ml to 5000.0 μg/ml), exposure time is from 10 seconds to 24 hours or from 24 hours to 365 days

24. The method according to claim 2, wherein the soil and/or water bodies in order to increase the productivity of agricultural and/or ornamental and/or forest and/or domestic plants and/or aquacultures are treated with the M4 in an amount from 0.001 μg/ml to 10e5 μg/ml or compositions M4 and zinc salts (from 0.1 μg/ml to 5000.0 μg/ml) glycerol (from 0.1 μg/ml to 5000.0 μg/ml) sorbitol (from 0.1 μg/ml to 5000.0 μg/ml), boron salts (from 0.1 μg/ml to 5000.0 μg/ml), glutamate salts (from 0.1 μg/ml to 5000.0 g/ml), mannitol (from 0.1 μg/ml to 5000.0 μg/ml), hydrogen phosphate salts (from 0.1 μg/ml to 5000.0 μg/ml), salts of dihydrophosphates (from 0.1 μg/ml to 5000.0 μg/ml), manganese salts (from 0.1 μg/ml to 5000.0 μg/ml), the exposure time is from 10 seconds to 24 hours and after exposure, the product is inactivated by adding carboxymethylcellulose and/or sodium alginate in a ratio with the products 0.5-1.0 to 100.0-1.0

25. The method according to claim 2, wherein reservoirs with running water are treated with products and the addition of the product ensures that the required final concentration of the M4 is maintained in an amount from 0.001 μg/ml to 10e5 μg/ml or compositions M4 and zinc salts (from 0.1 μg/ml to 5000.0 μg/ml) glycerol (from 0.1 μg/ml to 5000.0 μg/ml) sorbitol (from 0.1 μg/ml to 5000.0 μg/ml), boron salts (from 0.1 μg/ml to 5000.0 μg/ml), salts glutamate (from 0.1 μg/ml to 5000.0 μg/ml), mannitol (from 0.1 μg/ml to 5000.0 μg/ml), salts of hydrogen phosphates (from 0.1 μg/ml to 5000.0 μg/ml), salts of dihydrophosphates (from 0.1 μg/ml to 5000.0 μg/ml) manganese salts (from 0.1 μg/ml to 5000.0 μg/ml)

26. The method according to claim 2, wherein exposure to plants, root and/or non-root method, and/or spraying for application to the surface of vegetative shoots—leaves and stems and/or hydroponics and/or fertigation, for which M4 is used in an amount of 0.001 μg/ml to 10e5 μg/ml or compositions M4 and zinc salts (from 0.1 μg/ml to 5000.0 μg/ml) glycerol (from 0.1 μg/ml to 5000.0 μg/ml) sorbitol (from 0.1 μg/ml to 5000.0 μg/ml), boron salts (from 0.1 μg/ml to 5000.0 μg/ml), glutamate salts (from 0.1 μg/ml to 5000.0 μg/ml), mannitol (from 0.1 μg/ml to 5000.0 μg/ml), hydrogen phosphate salts (from 0.1 μg/ml to 5000.0 μg/ml), dihydrophosphate salts (from 0.1 μg/ml to 5000.0 μg/ml) manganese salts (from 0.1 μg/ml to 5000.0 μg/ml)

27. The method according to claim 2, for exposure to plants, root and/or foliar method, and/or spraying for application to the surface of vegetative shoots, leaves and stems and/or hydroponics and/or fertigation, for which M4 is used in an amount of 0.001 μg/ml to 10e5 μg/ml or compositions of M4 and zinc salts (from 0.1 μg/ml to 5000.0 μg/ml) glycerol (from 0.1 μg/ml to 5000.0 μg/ml) sorbitol (from 0.1 μg/ml to 5000.0 μg/ml), boron salts (from 0.1 μg/ml to 5000.0 μg/ml), glutamate salts (from 0.1 μg/ml to 5000.0 μg/ml), mannitol (from 0.1 μg/ml to 5000.0 μg/ml), hydrogen phosphate salts (from 0.1 μg/ml to 5000.0 μg/ml), salts of dihydrophosphates (from 0.1 μg/ml to 5000.0 μg/ml) of manganese salts (from 0.1 μg/ml to 5000.0 μg/ml), followed by inactivation with carboxymethyl cellulose, sodium alginate in ratio with the products 0.5-1.0 to 100.0-1.0

28. The method according to claim 2, to influence the eggs and fish eggs, to increase the efficiency of fertilization, and to control sex, the M4 is used in an amount from 0.001 μg/ml to 10e5 μg/ml or compositions M4 and zinc salts (from 0.1 μg/ml to 5000.0 μg/ml) glycerol (from 0.1 μg/ml to 5000.0 μg/ml) sorbitol (from 0.1 μg/ml to 5000.0 μg/ml), boron salts (from 0.1 μg/ml to 5000.0 μg/ml), glutamate salts (from 0.1 μg/ml to 5000.0 μg/ml), mannitol (from 0.1 μg/ml to 5000.0 μg/ml), salts of hydrogen phosphates (from 0.1 μg/ml to 5000.0 μg/ml), salts of dihydrophosphates (from 0.1 μg/ml to 5000.0 μg/ml) manganese salts (from 0.1 μg/ml to 5000.0 μg/ml), or reverse transcription inhibitors (from 0.1 μg/ml to 5000.0 μg/ml), integration/recombination (from 0.1 μg/ml to 5000.0 μg/ml), proteases (from 0.1 μg/ml to 5000.0 μg/ml), DNAse (from 0.1 μg/ml to 500.0 μg/ml), RNAse (from 0.1 μg/ml to 500.0 μg/ml), DNAse+RNAse (from 0.1 μg/ml to 500.0 μg/ml) and complexes of the listed products and the treatment lasts from 3 seconds to 1 hour or from 1 hour to 120 hours.

29. The method according to claim 2, for the effect on fish and/or crustaceans and/or molluscs to increase productivity, stress tolerance, increase of the growth rate, M4 is used in an amount from 0.001 μg/ml to 10e5 μg/ml or compositions M4 and zinc salts (from 0.1 μg/ml up to 5000.0 μg/ml) glycerol (from 0.1 μg/ml to 5000.0 μg/ml) sorbitol (from 0.1 μg/ml to 5000.0 μg/ml), boron salts (from 0.1 μg/ml to 5000.0 μg/ml), glutamate salts (from 0.1 μg/ml to 5000.0 μg/ml), mannitol (from 0.1 μg/ml to 5000.0 μg/ml), salts of hydrogen phosphates (from 0.1 μg/ml to 5000.0 μg/ml), salts of dihydrogen phosphates (from 0.1 μg/ml up to 5000.0 μg/ml) of manganese salts (from 0.1 μg/ml to 5000.0 μg/ml), and the treatment lasts from 3 seconds to 24 hours or from 24 hours to 120 hours

30. The method according to claim 2, for the effect on fish and/or crustaceans and/or molluscs to increase productivity, stress tolerance, increase of the growth rate, with M4 in an amount from 0.001 μg/ml to 10e5 μg/ml or compositions M4 and zinc salts (from 0.1 μg/ml to 5000.0 μg/ml) glycerol (from 0.1 μg/ml to 5000.0 μg/ml) sorbitol (from 0.1 μg/ml to 5000.0 μg/ml), boron salts (from 0.1 μg/ml to 5000.0 μg/ml), glutamate salts (from 0.1 μg/ml to 5000.0 μg/ml), mannitol (from 0.1 μg/ml to 5000.0 μg/ml), salts of hydrogen phosphates (from 0.1 μg/ml to 5000.0 μg/ml), salts of dihydrogen phosphates (from 0.1 μg/ml to 5000.0 μg/ml) of manganese salts (from 0.1 μg/ml to 5000.0 μg/ml), is introduced into the water where the fish are located and remains there indefinitely and/or is inactivated by carboxymethyl cellulose, sodium alginate in a ratio with the product 0.5-1, 0 to 100.0-1.0

31. The method according to P1.2 of exposure to fish and/or crustaceans and/or molluscs, which includes their treatment with the product, and release into water, also pre-treated with the product.

32. The method according to claim 2, in which to increase the growth rate, weight gain, and other vital and commercially important characteristics of animals and aquaplankton, the feed is treated before feeding with M4 in an amount from 0.001 μg/ml to 10e5 μg/ml or compositions M4 and zinc salts (from 0.1 μg/ml to 5000.0 μg/ml) glycerol (from 0.1 μg/ml to 5000.0 μg/ml) sorbitol (from 0.1 μg/ml to 5000.0 μg/ml), boron salts (from 0.1 μg/ml up to 5000.0 μg/ml), glutamate salts (from 0.1 μg/ml to 5000.0 μg/ml), mannitol (from 0.1 μg/ml to 5000.0 μg/ml), hydrogen phosphate salts (from 0.1 μg/ml to 5000.0 μg/ml), salts of dihydrophosphates (from 0.1 μg/ml to 5000.0 μg/ml) of manganese salts (from 0.1 μg/ml to 5000.0 μg/ml).

33. The method according to claim 2, wherein to increase the growth rate, weight gain, and other vital and commercially important characteristics of animals and aquaplankton, feed is treated before feeding with M4 in an amount from 0.001 μg/ml to 10e5 μg/ml or M4 compositions and zinc salts (from 0.1 μg/ml to 5000.0 μg/ml) glycerol (from 0.1 μg/ml to 5000.0 μg/ml) sorbitol (from 0.1 μg/ml to 5000.0 μg/ml), boron salts (from 0.1 μg/ml up to 5000.0 μg/ml), glutamate salts (from 0.1 μg/ml to 5000.0 μg/ml), mannitol (from 0.1 μg/ml to 5000.0 μg/ml), hydrogen phosphate salts (from 0.1 μg/ml to 5000.0 μg/ml), salts of dihydrophosphates (from 0.1 μg/ml to 5000.0 μg/ml) of manganese salts (from 0.1 μg/ml to 5000.0 μg/ml), followed by inactivation with carboxymethyl cellulose, sodium alginate in a ratio with the products 0.5-1.0 to 100, 0-1.0

34. The method according to claim 2, wherein to increase germination, growth intensity, chlorophyll formation and yield when pelleting seeds for 3 seconds to 24 hours, treatment is carried out using the M4 in an amount from 0.001 μg/ml to 10e5 μg/ml or compositions M4 and zinc salts (from 0.1 μg/ml to 5000.0 μg/ml) glycerol (from 0.1 μg/ml to 5000.0 μg/ml) sorbitol (from 0.1 μg/ml to 5000.0 μg/ml), boron salts (from 0.1 μg/ml to 5000.0 μg/ml), glutamate salts (from 0.1 μg/ml to 5000.0 μg/ml), mannitol (from 0.1 μg/ml to 5000.0 μg/ml), hydrogen phosphate salts (from 0.1 μg/ml to 5000.0 μg/ml), salts of dihydrophosphates (from 0.1 μg/ml to 5000.0 μg/ml) of manganese salts (from 0.1 μg/ml to 5000.0 μg/ml), or reverse transcription inhibitors (from 0.1 μg/ml to 5000.0 g/ml), integration/recombination (from 0.1 μg/ml to 5000.0 μg/ml), proteases (from 0.1 μg/ml to 5000.0 μg/ml), DNAse (from 0.1 μg/ml to 500.0 μg/ml), RNAse (from 0.1 μg/ml to 500.0 μg/ml), DNAse+RNAse (from 0.1 μg/ml to 500.0 m kg/ml) and complexes of the listed products

35. The method according to claim 2, wherein to increase germination, growth intensity, chlorophyll formation and yield when pelleting seeds for 3 seconds to 24 hours, treatment is carried out using the M4 in an amount from 0.001 μg/ml to 10e5 μg/ml or compositions M4 and zinc salts (from 0.1 μg/ml to 5000.0 μg/ml) glycerol (from 0.1 μg/ml to 5000.0 μg/ml) sorbitol (from 0.1 μg/ml to 5000.0 μg/ml), boron salts (from 0.1 μg/ml to 5000.0 μg/ml), glutamate salts (from 0.1 μg/ml to 5000.0 μg/ml), mannitol (from 0.1 μg/ml to 5000.0 μg/ml), hydrogen phosphate salts (from 0.1 μg/ml to 5000.0 μg/ml), salts of dihydrophosphates (from 0.1 μg/ml to 5000.0 μg/ml) of manganese salts (from 0.1 μg/ml to 5000.0 μg/ml), followed by inactivation with carboxymethyl cellulose, sodium alginate in a ratio with the product 0.5-1.0 up to 100.0-1.0 or reverse transcription inhibitors (from 0.1 μg/ml to 5000.0 μg/ml), integration/recombination (from 0.1 μg/ml to 5000.0 μg/ml), proteases (from 0.1 μg/ml to 5000.0 μg/ml), DNAse (from 0.1 μg/ml to 500.0 μg/ml), RNAse (from 0.1 μg/ml to 500.0 μg/ml), DNAse+RNAse (from 0.1 μg/ml to 500.0 μg/ml) and complexes of the above products

36. The method according to claim 2, to increase germination, growth rate, chlorophyll formation and yield, seed dressing is performed for 1.0 minutes to 24 hours using M4 in an amount from 0.001 μg/ml to 10e5 μg/ml or compositions M4 and zinc salts (from 0.1 μg/ml to 5000.0 μg/ml) glycerol (from 0.1 μg/ml to 5000.0 μg/ml) sorbitol (from 0.1 μg/ml to 5000.0 μg/ml), boron salts (from 0.1 μg/ml up to 5000.0 μg/ml), glutamate salts (from 0.1 μg/ml to 5000.0 μg/ml), mannitol (from 0.1 μg/ml to 5000.0 μg/ml), hydrogen phosphate salts (from 0.1 μg/ml to 5000.0 μg/ml), salts of dihydrophosphates (from 0.1 μg/ml to 5000.0 μg/ml), manganese salts (from 0.1 μg/ml to 5000.0 μg/ml), or reverse transcription inhibitors (from 0.1 μg/ml to 5000.0 μg/ml), integration/recombination (from 0.1 μg/ml to 5000.0 μg/ml), proteases (from 0.1 μg/ml to 5000.0 μg/ml), DNAse (from 0.1 μg/ml to 500.0 μg/ml), RNAse (from 0.1 μg/ml to 500.0 μg/ml), DNAse+RNAse (from 0.1 μg/ml to 500.0 μg/ml) and cops of the listed products

37. The method according to claim 2, wherein to increase germination, growth intensity, chlorophyll formation and yield, seed dressing is performed for 1.0 minutes to 24 hours using M4 in an amount of 0.001 μg/ml to 10e5 μg/ml or compositions M4 and zinc salts (from 0.1 μg/ml to 5000.0 μg/ml) glycerol (from 0.1 μg/ml to 5000.0 μg/ml) sorbitol (from 0.1 μg/ml to 5000.0 μg/ml), boron salts (from 0.1 μg/ml up to 5000.0 μg/ml), glutamate salts (from 0.1 μg/ml to 5000.0 μg/ml), mannitol (from 0.1 μg/ml to 5000.0 g/ml), hydrogen phosphate salts (from 0.1 μg/ml to 5000.0 μg/ml), salts of dihydrophosphates (from 0.1 μg/ml to 5000.0 μg/ml) of manganese salts (from 0.1 μg/ml to 5000.0 g/ml), followed by inactivation with carboxymethyl cellulose, sodium alginate in a ratio with the products 0.5-1.0 to 100, 0-1.0 or reverse transcription inhibitors (from 0.1 μg/ml to 5000.0 μg/ml), integration/recombination (from 0.1 μg/ml to 5000.0 μg/ml), proteases (from 0.1 μg/ml to 5000.0 μg/ml), DNAse (from 0.1 μg/ml to 500.0 μg/ml), RNAse (from 0.1 μg/ml to 500.0 μg/ml), DNAse+RNAse (from 0.1 μg/ml to 500.0 μg/ml) and complexes of the above products

38. The method according to claim 2, wherein to increase germination, growth intensity, chlorophyll formation and yield, seed dressing is performed for 1.0 minutes to 24 hours using M4 in an amount from 0.001 μg/ml to 10e5 μg/ml or compositions M4 and zinc salts (from 0.1 μg/ml to 5000.0 μg/ml) glycerol (from 0.1 μg/ml to 5000.0 μg/ml) sorbitol (from 0.1 μg/ml to 5000.0 μg/ml), boron salts (from 0.1 μg/ml up to 5000.0 μg/ml), glutamate salts (from 0.1 μg/ml to 5000.0 μg/ml), mannitol (from 0.1 μg/ml to 5000.0 μg/ml), hydrogen phosphate salts (from 0.1 μg/ml to 5000.0 μg/ml), salts of dihydrophosphates (from 0.1 μg/ml to 5000.0 μg/ml), manganese salts (from 0.1 μg/ml to 5000.0 μg/ml), or reverse transcription inhibitors (from 0.1 μg/ml to 5000.0 μg/ml), integration/recombination (from 0.1 μg/ml to 5000.0 μg/ml), proteases (from 0.1 μg/ml to 5000.0 μg/ml), DNAse (from 0.1 μg/ml to 500.0 μg/ml), RNAse (from 0.1 μg/ml to 500.0 μg/ml), DNAse+RNAse (from 0.1 μg/ml to 500.0 μg/ml) and cops of the listed followed by planting in soil pre-treated with the same products

39. The method according to claim 2, wherein for the bioactivation of the prevention and treatment of diseases of bees and increasing the amount of honey obtained, the hive is treated and fed with M4 in an amount from 0.001 μg/ml to 10e5 μg/ml or compositions M4 and zinc salts (from 0.1 μg/ml to 5000.0 μg/ml) glycerol (from 0.1 μg/ml to 5000.0 μg/ml) sorbitol (from 0.1 μg/ml to 5000.0 μg/ml), boron salts (from 0.1 μg/ml to 5000.0 μg/ml), glutamate salts (from 0.1 μg/ml to 5000.0 μg/ml), mannitol (from 0.1 μg/ml to 5000.0 μg/ml), hydrogen phosphate salts (from 0.1 μg/ml to 5000.0 μg/ml), dihydrogen phosphate salts (from 0.1 μg/ml to 5000.0 μg/ml) manganese salts (from 0.1 μg/ml to 5000.0 μg/ml), or reverse transcription inhibitors (from 0.1 μg/ml to 5000.0 μg/ml), integration/recombination (from 0.1 μg/ml up to 5000.0 μg/ml), proteases (from 0.1 μg/ml to 5000.0 μg/ml), DNAse (from 0.1 μg/ml to 500.0 μg/ml), RNAse (from 0.1 μg/ml to 500.0 μg/ml), DNAse+RNAse (from 0.1 μg/ml to 500.0 μg/ml) and complexes of the above products

40. The method according to claim 2, wherein correcting the condition of the skin and/or subcutaneous tissue of animals and humans in various diseases, including seborrheic dermatitis, neuroderma, Psoriasis, Herpes, papillomatosis, mycoses, erysipelas, trophic and diabetic ulcers, trauma, acne bedsores, folliculitis, furunculosis, angulitis (jam) alopecia using M4 in an amount from 0.001 μg/ml to 10e5 μg/ml or compositions M4 and zinc salts (from 0.1 μg/ml to 5000.0 μg/ml) glycerol (from 0.1 μg/ml to 5000.0 μg/ml) sorbitol (from 0.1 μg/ml to μg/ml), mannitol (from 0.1 μg/ml to 5000.0 μg/ml), hydrogen phosphate salts (from 0.1 μg/ml to 5000.0 μg/ml), dihydrophosphate salts (from 0.1 μg/ml to 5000.0 μg/ml), manganese salts (from 0.1 μg/ml to 5000.0 μg/ml), hyaluronic acid salts or reverse transcription inhibitors (from 0.1 μg/ml to 5000.0 μg/ml), integration/recombination (from 0.1 μg/ml to 5000.0 μg/ml), proteases (from 0.1 μg/ml to 5000.0 μg/ml), DNAse (from 0.1 μg/ml to 500.0 μg/ml), RNAse (from 0.1 μg/ml d about 500.0 μg/ml), DNAse+RNAse (from 0.1 μg/ml to 500.0 μg/ml) and complexes of the above products, while the products are gels and/or emulsions and/or ointments and/or liquids

41. The method according to claim 2, wherein correcting the state of the mucous membranes of the sinuses and/or the mucous membranes of the urinary bladder, in which using the product M4 in an amount from 0.001 μg/ml to 10e5 μg/ml or compositions M4 and zinc salts (from 0.1 μg/ml to 5000.0 μg/ml) glycerol (from 0.1 μg/ml to 5000.0 μg/ml) sorbitol (from 0.1 μg/ml to μg/ml), mannitol (from 0.1 μg/ml to 5000.0 μg/ml), hydrogen phosphate salts (from 0.1 μg/ml up to 5000.0 μg/ml), dihydrophosphate salts (from 0.1 μg/ml to 5000.0 μg/ml), manganese salts (from 0.1 μg/ml to 5000.0 μg/ml), hyaluronic acid salts or reverse transcription inhibitors (from 0.1 μg/ml up to 5000.0 μg/ml), integration/recombination (from 0.1 μg/ml to 5000.0 g/ml), proteases (from 0.1 μg/ml to 5000.0 μg/ml), DNAse (from 0.1 μg/ml to 500.0 μg/ml), RNAse (from 0.1 μg/ml to 500.0 μg/ml), DNAse+RNAse (from 0.1 μg/ml to 500.0 μg/ml) and complexes of the above products, while the products are gels and/or emulsions and/or liquid, in which the product is injected into the cavity for processing tissue and is either removed after washing or inactivated or remains in the cavity and endodontics, as well as cysts and granulomas using M4 in an amount from 0.001 μg/ml to 10e5 μg/ml or compositions M4 and zinc salts (from 0.1 μg/ml to 5000.0 μg/ml) glycerol (from 0.1 μg/ml to 5000.0 μg/ml) sorbitol (from 0.1 μg/ml to μg/ml), mannitol (from 0.1 μg/ml to 5000.0 μg/ml), salts of hydrogen phosphates (from 0.1 μg/ml to 5000.0 μg/ml), salts of dihydrophosphates (from 0.1 μg/ml to 5000.0 μg/ml), manganese salts (from 0.1 μg/ml to 5000.0 μg/ml), hyaluronic acid salts or reverse transcription inhibitors (from 0.1 μg/ml to 5000.0 μg/ml), integration/recombination (from 0.1 μg/ml to 5000.0 μg/ml), proteases (from 0.1 μg/ml to 5000.0 μg/ml), DNAse (from 0.1 μg/ml to 500.0 μg/ml), RNAse (from 0.1 μg/ml up to 500.0 μg/ml), DNAse+RNAse (from 0.1 μg/ml to 500.0 μg/ml) and complexes of the above products, while the products are gels and/or emulsions and/or liquids,

42. The method according to claim 2, wherein correction for correcting the condition of the eyes, including conjunctivitis and dacryocystitis, using the M4 in an amount from 0.001 μg/ml to 10e5 μg/ml or compositions M4 and zinc salts (from 0.1 μg/ml to 5000.0 μg/ml) glycerol (from 0.1 μg/ml to 5000.0 μg/ml) sorbitol (from 0.1 μg/ml to μg/ml), mannitol (from 0.1 μg/ml to 5000.0 μg/ml), hydrogen phosphate salts (from 0.1 μg/ml up to 5000.0 μg/ml), dihydrophosphate salts (from 0.1 μg/ml to 5000.0 μg/ml), manganese salts (from 0.1 μg/ml to 5000.0 μg/ml), hyaluronic acid salts or reverse transcription inhibitors (from 0.1 μg/ml up to 5000.0 μg/ml), integration/recombination (from 0.1 μg/ml to 5000.0 g/ml), proteases (from 0.1 μg/ml to 5000.0 μg/ml), DNAse (from 0.1 μg/ml to 500.0 μg/ml), RNAse (from 0.1 μg/ml to 500.0 μg/ml), DNAse+RNAse (from 0.1 μg/ml to 500.0 μg/ml) and complexes of the above products, while the products are gels and/or emulsions and/or liquids.
